(19) United States
(12) Patent Application Publication
(10) Pub. No.: US 2026/0021122 A1
(43) Pub. Date: Jan. 22, 2026

(54) CANNABINOIDS AND USES THEREOF IN TREATMENT OF A DISEASE

(71) Applicant: TECHNION RESEARCH & DEVELOPMENT FOUNDATION LIMITED, Haifa (IL)

(72) Inventors: David MEIRI, Haifa (IL); Iris WYROBNIK, Haifa (IL); Hila NOVAK KOTZER, Haifa (IL); Miryam STEINBERG, Haifa (IL); Anat GELFAND, Haifa (IL); Shiri PROCACCIA, Haifa (IL)

(21) Appl. No.: 19/103,611

(22) PCT Filed: Aug. 14, 2023
(86) PCT No.: PCT/IL2023/050850

§ 371 Date: Feb. 13, 2025

Related U.S. Application Data
(60) Provisional application No. 63/397,863, filed on Aug. 14, 2022.

(51) Int. Cl. A61K 31/00; A61K 36/185; A61K 45/06; A61P 35/00

(52) U.S. Cl. CPC A61K 31/658; A61K 36/3482; A61K 45/06; A61P 35/00

(57) ABSTRACT

The present invention provides a pharmaceutical composition including cannabigcrol. and methods of using same, such as for increasing therapeutic efficacy of an immune checkpoint inhibitor, or for the treatment of cancer.

### CROSS REFERENCE TO RELATED APPLICATIONS

This application claims the benefit of priority of U.S. Provisional Patent Application No. 63/397,863, filed Aug. 14, 2022, the content of which are incorporated herein by reference in its entirety.

### FIELD OF INVENTION

The present disclosure relates to pharmaceutical compositions comprising at least one cannabinoid, methods of use and methods of treatment thereof.

### BACKGROUND

The tumor microenvironment evolves during solid tumor progression into a highly immunosuppressive milieu. Key players in the formation of this anti-inflammatory environment are regulatory myeloid cells, such as Tumor-promoting (M2-like) tumor-associated macrophages and Myeloid-derived suppressor cells (MDSCs). Regulatory myeloid cells are recruited, polarized, and activated through tumor-secreted cytokines and chemokines, such as colony-stimulating factor 1 (CSF-1, also known as M-CSF), monocyte chemoattractant protein-1 (MCP-1, also known as CCL2) and macrophage inflammatory protein-2 (MIP-2, also known as CXCL2), which modulate the regulatory myeloid subpopulations. For example, CSF-1 is one of the cytokines that prevent MDSCs differentiation into mature myeloid cells and under pathological conditions leads to the expression of the M2 transcriptome. Additionally, blocking of the CSF-1 receptor was shown to stop the accumulation of immunosuppressive M2-like macrophages.

The generation and expansion of regulatory myeloid cells are one of the main mechanisms adopted by cancer cells to ensure tumor progression and metastasis formation. For example, once activated, MDSCs express various immunosuppressive markers, such as inducible nitrite oxide synthase (iNOS) and Arginase-1 (Arg-1), leading to reduced CD8+ T-cell proliferation and activation. One promising strategy in targeting regulatory myeloid cells is the depletion of tumor-secreted cytokines to achieve a reduction in their expansion and activation.

Naturally-occurring metabolites from the Cannabis plant possess the potential to modulate the suppressive functions of MDSCs. Medical Cannabis, and its unique metabolites known as phytocannabinoids, are gaining momentum in the field of drug development. They are under strong investigation as anti-cancer agents and the effect of phytocannabinoids on the immune system has been described previously. Phytocannabinoids modulate many processes in the body by their interaction with the endocannabinoid system, a ubiquitous and conserved neuromodulatory signaling system. The endocannabinoid system is involved in the regulation and proper functioning of most physiological systems, thus, phytocannabinoids have a huge potential to influence a variety of physiological processes and exert therapeutic effects in different diseases. The most commonly known cannabinoids, (−)-trans-Δ9-tetrahydrocannabinol (THC) and Cannabidiol (CBD), are extensively studied for therapeutic purposes, however, recent studies put the minor and less well-known phytocannabinoids into focus. The minor phytocannabinoids can be exploited in addition to the well-known major phytocannabinoids, as they were shown to exert a variety of effects in pathological conditions as well.

Most studies conducted in the cancer research field focused on how medical Cannabis affects cancer cells directly, without taking the complexity of the tumor ecosystem into consideration. A few studies focused on the effect of single cannabinoids on MDSCs, mostly THC or CBD, in healthy mice or immune-related diseases. However, it is still not clear how whole Cannabis extracts or minor cannabinoids affect regulatory myeloid cell populations and what role these cannabinoids play in the TME.

### SUMMARY

The present invention, in some embodiments, is based, at least in part, on the findings that altering the secretion of cytokines in the tumor microenvironment (TME) and thereby altering the characteristics of regulatory myeloid cells, immune evasion may be reduced, and tumor progression may be halted. The inventors show a novel mechanism by which medical Cannabis extracts and the single cannabinoid, Cannabigerol (CBG), modulate the differentiation and activation of regulatory myeloid cells leading to reduced cancer progression.

According to one aspect, there is provided a method for increasing the therapeutic efficacy of an immune checkpoint inhibitor in a subject treated therewith, the method comprising administering to the subject being treated with the immune checkpoint inhibitor a therapeutically effective amount of a pharmaceutical composition comprising cannabigerol (CBG) or any salt thereof, thereby increasing the therapeutic efficacy of an immune checkpoint inhibitor in the subject.

According to another aspect, there is provided a method for treating cancer in a subject in need thereof, the method comprising administering to the subject CBG and an immune checkpoint inhibitor, thereby treating cancer in the subject.

According to another aspect, there is provided a combination of CBG and an immune checkpoint inhibitor, for use in treatment of cancer in a subject in need thereof.

According to another aspect, there is provided a pharmaceutical composition comprising CBG and an immune checkpoint inhibitor, for use in treatment of cancer in a subject in need thereof.

According to another aspect, there is provided a kit comprising: (a) CBG; and (b) an immune checkpoint inhibitor.

In some embodiments, the subject is afflicted with cancer.

In some embodiments, the pharmaceutical composition consists essentially of CBG.

In some embodiments, the administering is at a synergistically effective amount.

In some embodiments, CBG is present as a highly purified extract of Cannabis.

In some embodiments, CBG is synthetically produced.

In some embodiments, treating comprises: reducing weight and/or volume of a tumor, increasing % of cancer cells undergoing apoptosis or cell death, reducing expression and/or secretion levels of colony stimulating factor 1 (CSF-1) from cancer cells, reducing number or abundance of myeloid-derived suppressor cells (MDSCs), M2-like tumor-associated macrophages, or both, increasing the number of cytotoxic CD8+ T cells, or any combination thereof, in the subject.

In some embodiments, the cytotoxic CD8+ T cells are expressing, secreting, or both, interferon gamma (INF-γ).

In some embodiments, the administering comprises administering CBG and immune checkpoint inhibitor separately or concomitantly.

In some embodiments, CBG is formulated within a first pharmaceutical composition and the immune checkpoint inhibitor is formulated within a second pharmaceutical composition.

In some embodiments, the kit further comprises instructions for: (i) mixing CBG and the immune checkpoint inhibitor; (ii) administering the mixed CBG and immune checkpoint inhibitor of (i) to a subject in need thereof; or (iii) both (i) and (ii).

In some embodiments, the kit is for increasing the therapeutic efficacy of an immune checkpoint inhibitor in a subject treated therewith, for treating cancer in a subject in need thereof, or both.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIGS. 1A-1H include graphs and micrograph showing that Cannabis extract treatment leads to a specific reduction in CSF-1 secretion by melanoma cells. (1A) WT C57B6/L female mice (8-10-weck-old, n=14) were subcutaneously injected with 1×10⁶ B16F10 cells. After 24 hr, mice were randomly divided into two groups and treated with either vehicle or the high-THC Cannabis chemovar CAN (100 mg/kg). After 14 days, the mice were sacrificed, and the tumors were excised and weighed. (1B) Representative image of excised tumors from mice treated with vehicle (top) or CAN (bottom). (1C) B16F10 cells were treated with a control (DMSO) or increasing concentrations of CAN extract for 24 hr (n=1-4). Cell death was evaluated by APC Annexin V/PI staining and apoptotic cells were measured using flow cytometry. (1D) B16F10 cells were treated with 2 μg/ml of CAN and assessed for cytokine secretion with the proteome profiler cytokine array. Representative membranes of the cytokine screen are depicted and four selected cytokines: CSF-1, MIP-2 CCL-5 and MCP-1 are shown in higher magnitude. (1E-1F) The concentrations (pg/ml) of MCP-1, MIP-2, and CCL5 (1E), and CSF-1 (1F) were measured using Enzyme-linked immunosorbent assay (ELISA) in tumor homogenates that originated from B16F10 tumors of mice treated over a period of 2 weeks with either Cannabis extract solvent (Control) or CAN (n=5-23/group). (1G-1H) Change in secreted CSF-1 concentration in the supernatants of B16F10 cells treated with 2 μug/ml (1G) and A375 (1H) treated with 6 μg/ml of CAN (n=4-6). Results were normalized to control group, presented as average +SEM and statistically analyzed by a student's t-test (NS—non-significant, *p<0.05, **p<0.01).

FIGS. 2A-2H include graphs and micrographs showing that cannabigerol (CBG) reduces CSF-1 secretion by B16F10 cells. (2A) Diagram of CAN chromatographic separation achieved using preparative HPLC/UV. CAN was separated into four fractions (F1-F4) according to their retention time using preparative HPLC/UV with a HALO CIS Fused-Core columon and a ternary A/B/C multistep hydrophobic gradient. (2B-2D) CSF-1 secretion was measured by ELISA and normalized to DMSO control after treatment of B16F10 cells with (2B) CAN and different combinations of fractions F1-F4 from CAN extract, (2C) CAN and different combinations of fractions F1-F2 and the synthetic cannabinoids CBN and CBG; and (2D) different ratios of CBG and F2. (2E) A375 cells were treated with 1.5 μg/ml CBG and 2 μg/ml 3704 and CSF-1 secretion was measured using ELISA. Results were normalized to DMSO. (F) CSF-1 protein expression in B16F10 cells treated with either DMSO control, 1.5 μg/ml CBG or 2 μg/ml 3704 was visualized by confocal microscopy using a 63× objective. The nuclei were visualized with DAPI. Relative AF-488 intensity of each treatment was normalized to the DMSO treatment. Each biological repeat included three random images taken, n=3. (2G) Representative images of F. (2H) B16F10 cells were treated as in E, then harvested with RIPA and the cell lysates were assessed for CSF-1 protein levels via western blotting with anti-CSF-1 and GAPDH as the loading control. Results are shown as fold-change+SEM, and statistical significance was calculated with one-way ANOVA (NS-non-significant, *p<0.05, **p<0.005, ***p<0.0005, ****p<0.0001).

FIGS. 3A-3G include a scheme and graphs showing that conditioned media (CM) from CBG-treated B16F10 cells reduces monocytic myeloid-derived suppressor cells (MO-MDSC) expansion and monocyte to macrophage transition. (3A) A non-limiting scheme showing experimental design of ex-vivo generation of BM-MDSCs from WT C57BL/6 mice. BMDCs were supplemented with 20 ng/ml of GM-CSF and IL-6 (4 days) to induce MDSC differentiation. Concurrently, B16F10 were treated with either DMSO, CBG (1.5 μg/ml) or the high-CBG extract 3704 (2 μg/ml) for 24 hours and the resulting conditioned media (CM) was used to treat the generated MDSCs. (3B) Fluorescence-activated cell sorting (FACS) gating strategy of monocytes, MO-MDSC and clear myeloid-derived suppressor cells (PMN-MDSC; black, orange and green rectangles, respectively). (3C-3E) The myeloid subpopulation frequencies of (3C) monocytes, (3D) MO-MDSCs and (3E) PMN-MDSCs were measured using flow cytometry after treatment for 24 hours of control growth media (no cells) with either DMSO, CBG or 3704, or CM from DMSO, CBG or 3704 treated B16F10 cells (n=6). (3F-3G) Summary of the MFI of macrophage marker F4/80 of Ly6C⁺ cells after 24 and 48 hours, respectively, with the indicated treatments (n=4). A representative histogram is shown on the left. Results are shown as average or fold-change±SEM and statistical significance was calculated by one-way ANOVA (NS-non-significant, *p<0.05, **p<0.01, ****p<0.0001).

FIGS. 4A-4G include graphs and a micrograph showing that MO-MDSCs show reduced inducible nitrite oxide synthase (INOS) expression leading to restored interferon gamma (INF-γ) expression by CD8+ T-cells when treated with CM from CBG- or 3704-treated B16F10 cells. (4A-4B) Percent of INOS expression (n=6) by MO-MDSCs (4A) and PMN-MDSCs (4B), representative flow charts are shown on the left. (4C) BM-MDSC were sorted 24 hours posttreatment into MO-MDSC and PMN-MDSC subpopulations. iNOS expression was measured by western blotting with INOS antibody and GAPDH as the loading control for each subpopulation separately. Representative blots are presented on the top and the relative intensity of three independent repeats is presented on the bottom. Results are presented as relative expression compared to MO-MDSCs treated with CM from DMSO-treated B16F10 cells. (4D-4G) Generated BM-MDSCs were treated with conditioned media for 24 hours, then sorted into MO-MDSC and PMN-MDSC subsets. The sorted MDSC subpopulations were co-cultured with activated CD8+ T-cells for 48 hours. Then CD8+ T-cells were prepared for intracellular staining and stained with IFN-γ-FITC. The frequencies of CD8+ IFN-γy+ cells were measured with flow cytometry and are presented with the matching contour plots for co-culture with MO-MDSCs (4D-4E) and PMN-MDSCs (4F-4G). Graphs are shown as average±SEM and statistically analyzed by one-way ANOVA (NS—non-significant, *p<0.05, **p<0.01, *****p<0.0001).

FIGS. 5A-5J include a diagram, graphs, and an image showing that synthetic CBG or high-CBG extract treatments reduce tumor progression and M2 macrophage frequencies in tumors. (5A) A non-limiting diagram of the mice model experiment created with Biorender. Female WT C57BL/6 mice (n=5/group) were injected with 1×10⁶ B16F10 cells. After 3 days, the mice were treated intraperitoneal with either vehicle, synthetic CBG (2.5 mg/kg), or the high-CBG extract 3704 (3.75 mg/kg), as indicated over the course of 14 days. (5B) Growth curve of ectopic tumor volume in mice. Tumors were measured using a vernier caliper and their volume was calculated according to the formula (length×width²)×0.5. (5C) Averaged tumor weight (grams) per treatment group on day 14. (5D) Image of excised tumors. (5E-5H) Single-cell suspensions were prepared from the excised tumors and myeloid cell frequencies were stained with extracellular markers for (E) CD45+ F4/80-Ly6C^{high} (F) CD45+ F4/80-Ly6G+ (G) CD45+ F4/80+Ly6C^{high} CD86+ (H) CD45+ F4/80+ Ly6C^{-/low} CD206+ and measured by spectral flow cytometry. (5I) M2/M1 frequency was calculated for each mouse and the average ratio is presented for the indicated groups. (5J) The single-cell suspension was stained with CD3+ and the frequency was measured by spectral flow cytometry. Statistically analyzed by two-way ANOVA for tumor volume (**p<0.005, ***p<0.0005, ****p<0.0001) or one-way ANOVA (NS-non-significant, *p<0.05).

FIGS. 6A-6B include vertical bar graphs showing cell death with increasing concentration of CAN, synthetic CBG, 3704 or CAN fractions. (6A-6B) Cell death percentage of (6A) A375 and (6B) B16F10 cells after 24 hours with the indicated treatments. Cells were stained with Fixable viability Dye and cell death percentage was measured using flow cytometry.

FIG. 7 includes scatter plots showing gating strategy of myeloid cell subpopulation from B16F10 tumors. FACS gating strategy of MO-MDSC and PMN-MDSC, M1 and M2 macrophages, and CD3+ T-cells. Analysis was conducted using the SpectroFlo® analysis software.

FIGS. 8A-8B include vertical bar graphs showing intracellular expression of Arg-1 in BM-MDSCs. (8A-8B) Percent of Arg-1 expression (n=2-4) by MO-MDSCs (8A) or PMN-MDSCs (8B). The cells were stained with the extracellular markers Ly6C. Ly6G and the intracellular marker Arg-1 and staining was measured using flow cytometry. Results are shown as mean±SEM and statistically analyzed with one-way ANOVA (NS—non-significant).

FIGS. 9A-9B include vertical bar graphs showing iNOS expression is unchanged in directly treated BM-MDSCs. (9A-9B) Percent of iNOS expression (n=4) by MO-MDSC (9A) and PMN-MDSC (9B). BM-MDSCs were generated as described and on day 4 medium from untreated B16F10 was added to cells together with either DMSO, 2 g/ml CBG or 6 μg/ml 3704 for 24 hours. Cells were stained with FVD for the exclusion of dead cells, and with the extracellular markers Ly6C, Ly6G and the intracellular marker iNOS. Staining was measured using flow cytometry. Results are shown as ±SEM and statistically analyzed with one-way ANOVA (NS—non-significant).

FIGS. 10A-10B include vertical bar graphs showing intracellular expression of GrzB in CD8+ T-cells co-cultured with MO-MDSCs or PMN-MDSCs. (10A-10B) Percent of GrzB expression in CD8+ T-cells co-cultured with (10A) MO-MDSCs or (10B) PMN-MDSCs. The cells were stained with the extracellular markers CD8 and the intracellular marker GrzB and staining was measured using flow cytometry. Results are shown as mean±SEM and statistically analyzed with one-way ANOVA (significance values are shown).

### DETAILED DESCRIPTION

The present invention provides cannabinoid compositions, plant extracts comprising cannabinoids, and methods of treating or ameliorating a disease using the described cannabinoid compounds, compositions, and extracts, in a subject in need thereof.

According to some embodiments, there is provided a method for increasing the therapeutic efficacy of an immune checkpoint inhibitor in a subject in need thereof.

According to some embodiments, there is provided a method for treating cancer in a subject in need thereof.

In some embodiments, the subject is afflicted with cancer. In some embodiments, the subject is treated with an immune checkpoint inhibitor. In some embodiments, the subject is treated for cancer using the immune checkpoint inhibitor. In some embodiments, the subject is administered with the immune checkpoint inhibitor for the treatment of cancer.

In some embodiments, the method comprises administering to the subject a therapeutically effective amount of a pharmaceutical composition comprising cannabigerol (CBG), thereby increasing the therapeutic efficacy of an immune checkpoint inhibitor in the subject.

As used herein, the terms “cannabigerol” and “CBG” include a compound represented by the formula:

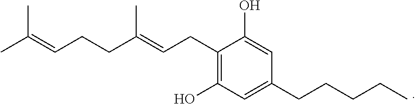 [SMILES: CCCCCc1cc(O)c(C/C=C(\C)CCC=C(C)C)c(O)c1]

or any pharmaceutically acceptable salt thereof.

In some embodiments, the pharmaceutical composition consists of essentially of CBG.

As used herein, the phrases “consist essentially of” or “consisting essentially of” denote that a given compound or substance, e.g., CBG constitutes the vast majority of the active ingredient's portion or fraction of the composition, e.g., a pharmaceutical composition.

In some embodiments, consisting essentially of means that the CBG constitutes at least 95%, at least 98%, at least 99%, or at least 99.9% by weight, of the active ingredient(s) of the pharmaceutical composition, or any value and range therebetween. Each possibility represents a separate embodiment of the invention.

In some embodiments, consisting essentially of means that CBG constitutes at least 98%, at least 99%, or at least 99.9% by weight of the active ingredient(s) of the pharmaceutical composition, or any value and range there between. Each possibility represents a separate embodiment of the invention.

In some embodiments, the method comprises administering to the subject CBG and an immune checkpoint inhibitor, thereby treating cancer in the subject.

In some embodiments, administering is at a synergistically effective amount.

In some embodiments, the term “synergistically effective amount” comprises any w:w or M:M ratio of CBG and an immune checkpoint inhibitor, wherein the therapeutic activity of CBG and the immune checkpoint inhibitor combined is greater than the summation of the individual therapeutic activities of CBG and the immune checkpoint inhibitor.

In some embodiments, CBG is present as a highly purified extract of Cannabis.

In some embodiments, Cannabis plant comprises or is hemp.

In some embodiments, CBG is synthetically produced.

As used herein, the term “immune checkpoint inhibitor” refer to inhibitors of CTLA4 (cytotoxic T lymphocyte antigen-4), PD-1 (programmed cell death protein 1), PD-L1 (programmed cell death ligand 1), PD-L2 (programmed cell death ligand 2), PD-L3 (programmed cell death ligand 3), PD-L4 (programmed cell death ligand 4), LAG-3 (lymphocyte activation gene-3), and TIM-3 (T cell immunoglobulin and mucin protein-3). In some embodiments, the immune checkpoint inhibitor is a binding ligand of PD-1. In some embodiments, the immune checkpoint inhibitor is a binding ligand of CTLA-4.

PD-1 is a key immune checkpoint receptor expressed by activated T and B cells and mediates immunosuppression. PD-1 is a member of the CD28 family of receptors, which includes CD28, CTLA-4, ICOS, PD-1, and BTLA. The term “PD-1” as used herein includes human PD-1 (hPD-1), variants, isoforms, and species homologs of hPD-1, and analogs having at least one common epitope with hPD-1.

Various cell surface glycoprotein ligands for PD-1 have been identified, including PD-L1, PD-L2, PD-L3, and PD-L4, that are expressed on antigen-presenting cells as well as many human cancers and have been shown to downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The term “PD-L2” as used herein includes human PD-L2 (hPD-L2), variants, isoforms, and species homologs of hPD-L2, and analogs having at least one common epitope with hPD-L2. The term “PD-L3” as used herein includes human PD-L3 (hPD-L3), variants, isoforms, and species homologs of hPD-L3, and analogs having at least one common epitope with hPD-L3. The term “PD-L4” as used herein includes human PD-L4 (hPD-L4), variants, isoforms, and species homologs of hPD-L4, and analogs having at least one common epitope with hPD-L4.

CTLA-4 (cytotoxic T-lymphocyte-associated protein 4) is a protein receptor that, functioning as an immune checkpoint, downregulates the immune system. CTLA4 is found on the surface of T cells, is also a member of the immunoglobulin (Ig) superfamily; CTLA-4 comprises a single extracellular Ig domain. CTLA-4 transcripts have been found in T cell populations having cytotoxic activity, suggesting that CTLA-4 might function in the cytolytic response.

In some embodiments, the immune checkpoint inhibitor is an inhibitor of PD-1, PD-L1, PD-L2, PD-L3, PD-L4, CTLA-4, LAG3, B7-H3, B7-H4, KIR or TIM3. In some embodiments, the immune checkpoint inhibitor is a PD-1 inhibitor. In some embodiments, the immune checkpoint inhibitor is a binding ligand of PD-L1. In some embodiments, the immune checkpoint inhibitor is a PD-L1 inhibitor. In some embodiments, the immune checkpoint inhibitor is a PD-L2 inhibitor or a combined PD-L1/PD-L2 inhibitor. In some embodiments, the immune checkpoint inhibitor is a CTLA-4 inhibitor.

In some embodiments, the immune checkpoint inhibitor can be a small peptide agent that can inhibit T cell regulation function. In some embodiments, the immune checkpoint inhibitor can be a small molecule (e.g. less than 500 Daltons) that can inhibit T cell regulation function. In some embodiments, the immune checkpoint inhibitor can be a molecule providing co-stimulation of T-cell activation. In some embodiments, the immune checkpoint inhibitor can be a molecule providing co-stimulation of natural killer cell activation. In some embodiments, the immune checkpoint inhibitor can be an antibody. In some embodiments, the immune checkpoint inhibitor is a PD-1 antibody. In some embodiments, the immune checkpoint inhibitor is a PD-L1 antibody. In some embodiments, the immune checkpoint inhibitor is a PD-L2 antibody. In some embodiments, the immune checkpoint inhibitor is a PD-L3 antibody. In some embodiments, the immune checkpoint inhibitor is a PD-L4 antibody. In some embodiments, the immune checkpoint inhibitor is a CTLA-4 antibody. In some embodiments, the immune checkpoint inhibitor is an antibody of CTLA-4, LAG3, B7-H3, B7-H4, KIR, or TIM3.

The antibody can be selected from a-CD3-APC, a-CD3-APC-H7, a-CD4-ECD, (X-CD4-PB, a-CD8-PE-Cy7, a-CD-8-PerCP-Cy5.5, a-CD 1 lc-APC, a-CDI Ib-PE-Cy7, a-CD1 1b-AF700, a-CD 14-FITC, a-CD16-PB, a-CD 19-AF780, a-CD 19-AF700, a-CD20-PO, a-CD25-PE-Cy7, a-CD40-APC, a-CD45-Biotin, Streptavidin-BV605, a-CD62L-ECD, a-CD69-APC-Cy7, a-CD80-FITC, a-CD83-Biotin, Streptavidin-PE-Cy7, a-CD86-PE-Cy7, a-CD86-PE, a-CD123-PE, a-CD 154-PE, a-CD 161-PE, a-CTLA4-PE-Cy7, a-FoxP3-AF488 (clone 259D), IgG 1-isotype-AF488, a-ICOS (CD278)-PE, a-HLA-A2-PE, a-HLA-DR-PB, a-HLA-DR-PerCPCy5.5, a-PD 1-APC, VISTA, co-stimulatory molecule OX40, and CD 137.

A variety of antibodies (Abs) can be used in the methods, composition, combination therapy, or the kit, as described herein, including antibodies having high-affinity binding to PD-1 PD-L1, PD-L2, PD-L3, or PD-L4. Human mAbs (HuMAbs) that bind specifically to PD-1 (e.g., bind to human PD-1 and may cross-react with PD-1 from other species, such as cynomolgus monkey) with high affinity have been disclosed in U.S. Pat. No. 8,008,449, which is incorporated herein by reference in its entirety. HuMAbs that bind specifically to PD-L1 with high affinity have been disclosed in U.S. Pat. No. 7,943,743, which is incorporated herein by reference in its entirety. Other anti-PD-1 mAbs have been described in, for example, U.S. Pat. Nos. 6,808,710, 7,488,802 and 8,168,757, and PCT Publication No. WO 2012/145493, all of which are incorporated herein by reference in their entireties. Anti-PD-L1 mAbs have been described in, for example, U.S. Pat. Nos. 7,635,757 and 8,217,149, U.S. Publication No. 2009/0317368, and PCT Publication Nos. WO2011/066389 and WO 2012/14549, all of which are incorporated herein by reference in their entireties.

In some embodiments, the anti-PD-1 HuMAbs can be selected from 17D8, 2D3, 4H 1, 5C4 (also referred to herein as nivolumab), 4A 1 1, 7D3 and 5F4, all of which are described in U.S. Pat. No. 8,008,449. In some embodiments, the anti-PD-1 HuMAbs can be selected from 3G10, 12A4 (also referred to herein as BMS-936559), 10A5, 5F8, 10H10, 1B 12, 7H1, 1 1E6, 12B7, and 13G4, all of which are described in U.S. Pat. No. 7,943,743.

In some embodiments, treating comprises reducing weight and/or volume of a tumor, in a subject.

In some embodiments, treating comprises increasing % of cancer cells undergoing apoptosis or cell death, in a subject.

In some embodiments, treating comprises reducing expression and/or secretion levels of colony stimulating factor 1 (CSF-1) from cancer cells, in a subject.

In some embodiments, treating comprises reducing number or abundance of myeloid-derived suppressor cells (MDSCs), M2-like tumor-associated macrophages, or both, in a subject.

In some embodiments, treating comprises increasing the number of cytotoxic CD8+ T cells in a subject.

In some embodiments, cytotoxic CD8+ T cells are expressing, secreting, or both, interferon gamma (INF-γ).

In some embodiments, administering comprises administering CBG and an immune checkpoint inhibitor separately or concomitantly.

According to some embodiments, there is provided a combination of CBG and an immune checkpoint inhibitor, for use in treatment of cancer in a subject in need thereof.

In some embodiments, CBG is formulated within a first pharmaceutical composition and an immune checkpoint inhibitor is formulated within a second pharmaceutical composition.

According to some embodiments, there is provided a pharmaceutical composition comprising CBG and an immune checkpoint inhibitor, for use in treatment of cancer in a subject in need thereof.

In some embodiments, a pharmaceutical composition as disclosed herein, further comprises a pharmaceutically acceptable carrier, excipient, or diluent.

According to some embodiments, there is provided a kit comprising: (a) CBG; and (b) an immune checkpoint inhibitor.

In some embodiments, the kit further comprises instructions for: (i) mixing CBG and an immune checkpoint inhibitor; (ii) administering a mixed or a mixture of CBG and an immune checkpoint inhibitor obtain according to (i) to a subject in need thereof; or (iii) both (i) and (ii).

In some embodiments, the kit is for increasing the therapeutic efficacy of an immune checkpoint inhibitor in a subject treated therewith.

In some embodiments, the kit is for treating cancer in a subject in need thereof.

In some embodiments, the kit is for increasing the therapeutic efficacy of an immune checkpoint inhibitor in a subject treated therewith and for treating cancer in a subject in need thereof.

In some embodiments, the present invention is directed, at least in part, to a composition derived from a plant extract. In some embodiments, a plant extract of the invention is derived from a plant comprising cannabinoids. In some embodiments, the plant extract of the invention is derived from a Cannabis plant. In some embodiments, the plant extract is derived from a specific species of the Cannabis genus.

In some embodiments, the pharmaceutical composition comprises one cannabinoid.

In some embodiments, the at least one cannabinoid is CBG.

In some embodiments, the composition further comprises one or more additional cannabinoids. According to some embodiments, the invention relates to a composition comprising a plurality of cannabinoids.

In some embodiments, the active agent of the pharmaceutical composition is or consists essentially of CBG.

In some embodiments, the subject is a human subject.

In some embodiments, the composition is a pharmaceutical composition.

In some embodiments, the composition comprises or consists of a plant extract.

As used herein, the term “extract” comprises the whole extract, a fraction thereof, a portion thereof, an isolated compound therefrom, or any combination thereof.

In some embodiments, the extract is derived from a plant material.

In some embodiments, the extract comprises an extract being derived from a high-CBG chemovar, termed ‘3704’.

In some embodiments, the plant material is first dried and then extracted. In some embodiments, the plant material is air-dried. In some embodiments, the plant material is further heat treated (e.g., hot-drying) and then extracted.

As used herein, treatment before extraction comprises, for example, freezing, drying, lyophilizing, or any combination thereof.

In some embodiments, the plant material is further processed prior to the extraction procedure in order to facilitate the extraction procedure. In some embodiments, processing methods prior to extraction, include but are not limited to crushing, slicing, or shredding, such as by using a grinder or other devices to fragment the plant parts into small pieces or powder.

In some embodiments, the extraction is a solvent-based extraction. In some embodiments, the solvent is a polar solvent. As used herein, a polar solvent may be selected from the group including, but not limited to, ethanol and Isopropyl. In some embodiments, the solvent is a non-polar solvent. In some embodiments, the extraction is a solvent-less-based extraction.

According to some embodiments, there is provided a pharmaceutical composition comprising the herein disclosed cannabinoids and a pharmaceutically acceptable carrier.

In some embodiments, the Cannabis derived substance used in the composition and methods as described herein comprises or consists essentially of CBG.

As used herein, the term “synthetic cannabinoids” refers to compounds that have a cannabinoid or cannabinoid-like structure and are manufactured using chemical means rather than by the plant. In some embodiments, a synthetic cannabinoid is or comprises a biosynthetic cannabinoid, such as, but not limited to, being produced by a cell or a culture under in vitro conditions.

As used herein, the term “carrier”, “excipient” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier (e.g. carbomer, hydroxypropyl cellulose, sodium lauryl sulfate) as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

A pharmaceutical composition may take any physical form necessary for proper administration. A composition comprising an encapsulated one or more cannabinoid compounds can be administered in any suitable form, including but not limited to a liquid form, a gel form, a semi-liquid (e.g., a liquid, such as a viscous liquid, containing some solid) form, a semi-solid (a solid containing some liquid) form, or a solid form. Compositions can be provided in, for example, a tablet form, a capsule form, a liquid form, a food form a chewable form, a non-chewable form, a transbuccal form, a sublingual form, a slow-release form, a non-slow-release form, a sustained release form, or a non-sustained-release form.

A pharmaceutically-acceptable carrier suitable for the preparation of unit dosage form of a composition as described herein for peroral administration is well-known in the art.

In some embodiments, the compositions further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmellose sodium, crospovidone, guar gum, sodium starch glycolate), additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), stabilizers (e.g. hydroxypropyl cellulose, hydroxypropylmethyl cellulose), viscosity increasing agents (e.g. carbomer, colloidal silicon dioxide, ethyl cellulose, guar gum lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), polymer coatings (e.g., poloxamers or poloxamines), and/or coating and film forming agents (e.g. ethyl cellulose, acrylates, polymethacrylates).

In some embodiments, preparation of effective amount or dose can be estimated initially from in vitro assays. In one embodiment, a dose can be formulated in animal models, and such information can be used to determine useful doses more accurately, in humans.

In one embodiment, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages vary depending upon the dosage form employed and the route of administration utilized. In one embodiment, the exact formulation, route of administration and dosage can be chosen by the individual physician in view of the patient's condition. [See e.g., Fingl, et al., (1975) “The Pharmacological Basis of Therapeutics”, Ch. 1 p. 1].

According to some embodiments, the method comprises treating or ameliorating cancer or a symptom associated therewith.

As used herein, the terms “administering”, “administration”, and like terms refer to any method which, in sound medical practice, delivers a composition containing an active agent to a subject in such a manner as to provide a therapeutic effect. One aspect of the present subject matter provides for dermal or transdermal administration of a therapeutically effective amount of a composition of the present subject matter to a subject in need thereof. Other suitable routes of administration can include oral, dermal, transdermal, parenteral, subcutaneous, intravenous, intramuscular, or intraperitoneal. In some embodiments, the administering is systemic administering. In some embodiments, the administering is to the inflamed skin site.

In some embodiments, compositions for use in the methods of this invention comprise solutions or emulsions, which in some embodiments are aqueous solutions or emulsions comprising a safe and effective amount of the cannabinoid of the present invention and optionally, other compounds as described herein.

In some embodiments, the composition is formulated with a carrier. In some embodiments, the composition is encapsulated.

In one embodiment, the amount of a composition to be administered will be dependent on the subject being treated, the severity of the affliction, the manner of administration, the judgment of the prescribing physician, etc.

The dosage administered will be dependent upon the age, health, and weight of the recipient, kind of concurrent treatment, if any, frequency of treatment, and the nature of the effect desired.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

As used herein, the term “about” when combined with a value refers to plus and minus 10% of the reference value. For example, a length of about 1,000 nanometers (nm) refers to a length of 1,000 nm±100 nm.

It is noted that as used herein and in the appended claims, the singular forms “a”, “an”, and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely”, “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Reagents

Synthetic CBG (Symrise, Germany, Cat #664714) and synthetic CBN (Open Book Extracts, Cat #IOB_JACGA_CBN) were diluted in DMSO (Merck Millipore, Cat #101900) to stock solutions.

### Cell Lines and Culture Conditions

The human melanoma cell line A375 was purchased from ATCC (Cat #CRL-1619). B16F10 cells were incubated in RPMI (Sigma, Cat #R8758) and A375 in DMEM (Sigma, Cat #D5796), supplemented with 10% FBS (Sigma, Cat #F7524) and 1% Penicillin/Streptavidin (Sartorius, Cat #03-031-1B). All cells were maintained in a humidified incubator at 37°° C. with 5% CO₂.

### Phytocannabinoid Extraction and Analysis

This study focused on two Cannabis extracts, the high-THC extract CAN and the high-CBG extract 3704. Air-dried medical Cannabis female inflorescences of these chemovars were ground to a fine powder using an electric grinder. Both samples were heat-decarboxylated in an oven at 130° C. for 1 h. Exactly 5 g from each chemovar were accurately weighed and extracted with 50 mL HPLC-grade ethanol. Samples were sonicated in an ultrasonic bath for 30 min and agitated in an orbital shaker at 25° C. for 15 min. Samples were then filtered under pressure through Whatman filter paper number 4 and the ethanol was evaporated under reduced pressure at 38° C. using a rotary evaporator.

Phytocannabinoids were analyzed by reversed-phase ultra-high-performance liquid chromatography with an ultraviolet detector (UHPLC/UV) system (Thermo Scientific, Bremen, Germany). The phytocannabinoid analytical standards (>98%) cannabigerol (CBG; C-141), 4⁹-THC (T-005), CBD (C-045), cannabichromene (CBC; C-143), cannabinol (CBN; C-046), cannabigerolic acid (CBGA; C-142), (−)-Δ⁹-trans-tetrahydrocannabinolic acid (Δ⁹-THCA; T-093), cannabidiolic acid (CBDA; C-144), cannabinolic acid (CBNA; C-153), cannabichromenic acid (CBCA; C-150), (−)-Δ⁸-trans-tetrahydrocannabinol (Δ⁸-THC; T-032), (−)-Δ⁹-trans-tetrahydrocannabivarin (Δ⁹-THCV; T-094), cannabidivarin (CBDV; C-140), cannabidivarinic acid (CBDVA; C-152), and cannabicyclol (CBL; C-154) were purchased from Sigma-Aldrich; cannabichromevarin (CBCV; 21974) and cannabicitran (21295) were purchased from Cayman Chemical Company.

The chromatographic separation was achieved using a HALO C18 Fused-Core column (2.7 μm, 150×2.1 mm i.d.), with a HALO guard column (2.1×5 mm, 2.7 μm), and a ternary A/B/C multistep gradient (solvent A: 0.1% acetic acid in water, solvent B: 0.1% acetic acid in acetonitrile, and solvent C: methanol, all solvents were of LC/MS grade). The multistep gradient program was established as follows: initial conditions were 50% B raised to 67% B until 3 min, held at 67% B for 5 min, and then raised to 90% B until 12 min, held at 90% B until 15 min, decreased to 50% B over the next min, and held at 50% B until 20 min for re-equilibration of the system prior to the next injection. Solvent C was initially 5% and then lowered to 3% until 3 min, held at 3% until 8 min, raised to 5% until 12 min and then kept constant at 5% throughout the run. A flow rate of 0.25 mL/min was used, the column temperature was 30° C. and the injection volume was 1 μL. Data acquisition was performed in full UV-Vis scan mode. Standard mixes in DMSO were prepared ranging from 1 to 1000 μg/mL for Δ⁹-THCA, Δ⁹-THC, CBDA and CBD, and 0.25 to 250 μg/mL for all the other components.

### Fractionation of Whole Extract

Fractionation of CAN extract into four fractions was performed using 1260 Infinity II LC System semi-preparative HPLC/UV (Agilent Technologies, CA, USA). The chromatographic separation was achieved using a Zorbax Eclipse XBD-C18 column (5 μm, 150 mm×30 mm i.d.) and a two-solvent A/B multistep gradient (solvent A: 0.05% formic acid in water and solvent B: 0.05% formic acid in acetonitrile, all HPLC grade). The multistep gradient program was established as follows: initial conditions were 65% B for 3 min, raised to 76% B until 12 min, then raised to 95% and held at 95% B for 6 min, and then decreased to 65% B over the next 3 min. A flow rate of 40 mL/min and an injection volume of 0.75 mL were used. Data acquisition was performed at 220 nm. The crude Cannabis extract was prepared in a concentration of 68 mg/mL in ethanol. Fractions were collected and then lyophilized to dryness and analyzed by UHPLC/UV and ESI-LC/MS. Fraction F2 contained 93% THC.

### Apoptosis Assay

Apoptosis was assessed by flow cytometry, measuring the percent of apoptotic cells stained in an annexin binding buffer (BioVision, Cat #1006-100) with APC Annexin V (BioLegend, Cat #640941, 1:100) and propidium iodide (PI, BioLegend, Cat #421301, 1:400) after 24 hours with the indicated treatments. Stained cells were analyzed by BD FlowJo™ software version x.0.7. (BD Biosciences). Cell death was determined as the percentage of positive APC Annexin V and/or double stained with PI out of total cells counted (20,000 events).

### Cytokine Screen

The media from treated B16F10 cells were collected, and the contents of cytokines and chemokines were measured using the proteome profiler mouse cytokine array kit panel A with 40 different cytokine antibodies (R&D Systems; Cat #ARY006) as described in the manufacturer's protocol.

### ELISA

The media from B16F10 and A375 cell lines, or homogenates from B16F10 tumors, that were treated as indicated for 24 hours, were collected and the protein concentration of MCP-1, MIP-2, C-C Motif Chemokine Ligand 5 (CCL5), and CSF-1 were measured by Quantikine Mouse M-CSF Immunoassay solid-phase ELISA kit (R&D systems, Cat #MMC00) as described in the manufacture's protocol.

### Immunofluorescence (IF) Microscopy

B16F10 cells were seeded on 13 mm coverslips (Bar Naor Ltd., Cat #BN1001-13-1CN) and treated with either synthetic CBG or Cannabis extract 3704 for 24 hours. Then, cells were fixed at room temperature with 4% PFA (Electron Microscopy Sciences, Cat #15710) in PBS (Sartorius, Cat #02-023-1A) for 20 minutes, permeabilized by 0.3% Triton-100× (Sigma, Cat #T8787) in PBS for 10 minutes and Blocked with a solution containing 5% Normal Goat Serum (Abcam, Cat #ab7481), 0.25% Gelatin (Sigma-Aldrich, Cat #G7041) and 0.1% Triton-X100 in PBS for 1 hour. Cells were then stained for 2 hours with CSF-1 (Santa Cruz; Clone D-4; Cat #365779, 1:50), washed and stained with the secondary antibody anti-mouse Alexa Flour 488-secondary antibody (Abcam, Cat #150077, 1:500) for one hour, fixed again with 4% PFA for 10 min and mounted with a mounting solution containing DAPI (GBI labs Cat #K1996619B). Samples were imaged using the Zeiss LSM 710 inverted confocal microscope with 20× (0.8NA) Plan-Apochromat lens for statistical analysis and Plan-Apochromat 63x/1.4 Oil objective lens for representative image acquisition. Analysis was conducted using the IMARIS software and results were calculated using the formula: mean AF-488 intensity divided by the number of nuclei.

### Mouse Tumor Model

All procedures involving animals were conducted in accordance with the standards approved by the Technion Animal Care and Use Ethics Committee and the studies adhered to the Technion Code for Experimentation on Animals. To induce tumors, B16F10 cells grown to a confluency of ˜75% were resuspended to a concentration of 1×10⁶ cells in 200 ul sterile PBS and transplanted subcutaneously into the right flank of 8-10-week-old female WT C57B6/L mice (Envigo, 16.8±0.78 g). After either 24 hours or 3 days, mice were randomly divided into groups and treated as indicated with either a vehicle of Cannabis extract solvent (18:1:1 ratio of DDW+0.9% NaCl, Sigma, Cat #S7653; Cremophor® EL, Cat #238470; Ethanol, Sigma, #Cat 111727), CAN (100 mg/kg), synthetic CBG (2.5 mg/kg) or 3704 (3.75 mg/kg) for 5 days, then 2 days off and again for 4 days. Tumor volume was measured from day 10 to 14 (endpoint) using a vernier caliper and calculated according to the formula (length×width²)^{0.5} and all tumors were weighed at the endpoint of the experiments with an analytical scale.

To prepare single-cell suspensions from the tumors, on day 14 the tumors were excised from the mice and placed in Hanks' Balanced Salt solution (HBSS, Sigma; Cat #H6648) containing 20 mg/ml DNase I (Sigma, Cat #D5025), 1 mg/ml collagenase D from Clostridium histolyticum (Sigma-Aldrich, Cat #C5138) and 0.1 mg/ml hyaluronidase (Sigma-Aldrich, Cat #6254). Tumors were mechanically digested using a scalpel and the gentleMACS™M Dissociator (Miltenyi Biotec). The resulting solution was filtered through a 100 μm filter (Alex Red Ltd. Cat #SO SCS1002) and centrifuged for 10 minutes at 400×g. The supernatant was further used for cytokine and chemokine concentration measurements. For the characterization of myeloid cell frequencies in tumors, the cell pellet was further processed as follows: red blood cells were lysed using the red blood lysis buffer (Biological industries, Cat #01-888-1B), which was neutralized by washing with PBS+2% FBS and centrifuging 10 minutes at 400×g. After centrifugation, cells were resuspended in 40% percoll gradient solution (prepared from 100% percoll, Cytiva, #17089101) and then 80% percoll was added on top of the 40% solution without mixing. The cell suspension was centrifuged at room temperature for 30 minutes without acceleration and break at 500×g. Cells were isolated from the interphase between the 40% and 80% percoll solutions and washed with PBS +2% FBS. Finally, cells were stained with extracellular markers for CD3+ T-cells and myeloid cells, and subpopulation frequency was measured by spectral flow cytometry using Cytek® Aurora (Cytek Biosciences).

### In-Vitro Bone-Marrow Derived MDSC (BM-MDSC) Differentiation

Bone-marrow-derived cells (BMDCs) were isolated from the fibula and tibia bones of healthy female WT C57BL/6 mice (8-12-week-old). Red blood cells were removed by using red blood lysis buffer (Biological industries, Cat #01-888-1B). Remaining cells were counted and 1×10⁶ cells were seeded in complete RPMI (Sigma, Cat #R8758) containing 10% FBS (Sigma, Cat #F7524), 1% L-Glutamine (Sartorius, Cat #03-020-1B), 1% Penicillin/Streptavidin (Sartorius, Cat #03-031-1B), 1% MEM-Eagle (Sartorius, Cat #01-340-1B), 2.5% HEPES (Sartorius, Cat #03-025-1B) and 1% sodium pyruvate (Sartorius, Cat #03-042-1B) and supplemented with 20 ng/ml of GM-CSF and IL-6 (R&D systems, 415-ML and 406-ML). Cells were incubated at 37° C. and 5% CO₂ for 4 days and then treated with conditioned media, which was the media collected from B16F10 cells treated with either DMSO, CBG, or the high-CBG extract 3704 as indicated.

### BM-MDSCs CD8+ T-Cell Co-Culture Suppression Assay

Generated bone marrow-MDSC (BM-MDSCs) were treated with conditioned media for 24 hours, then sorted into MO-MDSC and PMN-MDSC subsets. Concurrently, CD8+ T-cells were isolated from the spleens of healthy female WT C57BL/6 mice (8-12-weck-old). To isolate CD8+ T-cells, spleens were mechanically disintegrated and filtered through a 70 um filter (Alex Red Ltd., Cat #SO SCS702), the red blood cells were lysed with the red blood lysis buffer (Biological industries, Cat #01-888-1B). Then, CD8+ T-cells were isolated using the EasySep Mouse CD8+T-cell isolation kit (Stemcell Technologies, Cat # 19853) according to the manufacturer's protocol. The isolation purity was measured by staining cells before and after isolation with CD8-PECY7 (BioLegend, Cat #100722, Clone 53-6.7) by flow cytometry.

Post-sorted MDSC subpopulations were co-cultured in complete RPMI with CD8+ T-cells that are activated with Dynabeads® Mouse T-Activator CD3/CD28 kit (Cat # 11456D; 12.5 μl/1×10⁶ cells). After 48 hours, the activation beads were removed and cells were incubated with eBioscience Cell Stimulation Cocktail (Invitrogen, Cat #00-4970-93) for 3 hours and then also with BD GolgiStop™ Protein Transport Inhibitor (BD Biosciences, Cat #51-2092KZ) for the detection of IFN-γ. Intracellular staining of IFN-γ in CD8+ T-cells was achieved by using the BD Cytofix/Cytoperm™ Kit (BD Biosciences, Cat #554714) as stated in the manufacturer's instructions and then staining with IFN-γ-FITC (BioLegend, Cat #505806, Clone XMG1.2) and GranzymeB-FITC (BioLegend, Cat #372206, clone QA16A02).

### Flow Cytometry

To differentiate the subpopulation of myeloid cells, BMDCs and myeloid cells from tumors were stained for the extracellular makers Ly6C-BV421 (BioLegend, Clone HK1.4; Cat #128032) or Ly6C-FITC (BioLegend, Cat #128096, Clone HK1.4), Ly6G-APC (BioLegend, Clone 1A8; Cat #127614), Ly6G-BV421 (BioLegend, Cat #127627, clone 1A8), PECy7-F4/80 (BioLegend, Clone BM8 Cat #123113), BV605-CD86 (BioLegend, Clone GL-1, Cat #105037), APC-CD206 (Biolegend, Clone CO68C2, Cat #141708) and BV711-CD45 (BioLegend, Clone 30F11, Cat #103147). Cells from tumors were also stained with PE-CD3+ (BioLegend, Clone 17A2, Cat #100206) to identify the overall T-cell population. To characterize the cells in the subpopulations, cells were fixed and permeabilized with the Intracellular Fixation and Permeabilization Buffer Set (eBioscience Cat #88-8824-00) and stained with FITC-iNOS (Thermo, Clone CXNFT, Cat #53592082) or FITC-Arginase-1 (Thermo, Clone AlexF5, Cat #53-3697-82). These cells were also stained with Fixable viability Dye (FVD)-eflour 780 (Thermo, Cat #65086514) to exclude dead cells.

### Fluorescent Activated Cell Sorting (FACS)

To analyze the two subpopulations of BM-MDSCs, the cells were stained with Ly6C-BV421 and Ly6G-APC and sorted into MO-MDSC or PMN-MDSC using the BD FACSAria III Cell Sorter instrument (BD Biosciences). Directly after sorting, the two separate subpopulations were co-cultured with CD8+ T-cells or lysed and further analyzed via western blot.

### Preparation of Cell Lysates and Western Blot

B16F10 cells, and sorted MO-MDSC and PMN-MDSC achieved as described, were lysed in RIPA buffer (Sigma, Cat #R0278) supplemented with Protease/Phosphatase Inhibitor Cocktail (Cell signaling, Cat #5872S) on ice for 20 minutes. Total protein concentrations were measured using the detergent-compatible protein assay (Bio-Rad, Cat #5000113 and 5000114) and equal amounts were loaded onto a 4%-20% gradient gel (Invitrogen, Cat #XPO4202) and semidry transferred into a nitrocellulose membrane (Bio-Rad, Cat #1704159). The membrane was incubated with the primary antibodies CSF-1 (Santa Cruz; Clone D-4; #sc-365779), iNOS (Abcam; Clone #ab15323) or GAPDH (Cell Signaling Technology, Cat #14C10). Secondary antibodies used in this study were HRP-conjugated and the membranes were developed with the ECL detection kit (Millipore, Cat #WBLUR).

### Statistical Analysis

The data were analyzed by GraphPad Prism 6 (GraphPad Software Inc.) and the results were presented as mean±SEM. Comparisons were performed using one-or two-way ANOVA or a student's t-test as indicated. A p-value <0.05 was considered statistically significant.

### Example 1

### CSF-1 Secretion is Reduced After Treatment with a High-THC Cannabis Extract

The inventors have previously shown that Cannabis extracts differ greatly in their phytocannabinoid profile and therefore in their anticancer activity. In those studies, the inventors observed that some Cannabis extracts exert apoptotic effects while others influenced the proliferation or the dormant state of cancer cells. Myeloid cells were found to play key roles in the dormancy state of cancer cells. Therefore, in the current study the inventors focused on the effect of Cannabis, and its bioactive metabolites, the cannabinoids, on the characteristics of myeloid cells in the tumor microenvironment. Previous studies established that tumor-secreted cytokines lead to altered myeloid cell differentiation into immunosuppressive regulatory cells and activate them in the tumor microenvironment. The murine melanoma cell line B16F10 is intensively used for studying the interaction between cancer cells and regulatory myeloid cells since they secrete a great variety of cytokines and chemokines, therefore the inventors focused on these cells.

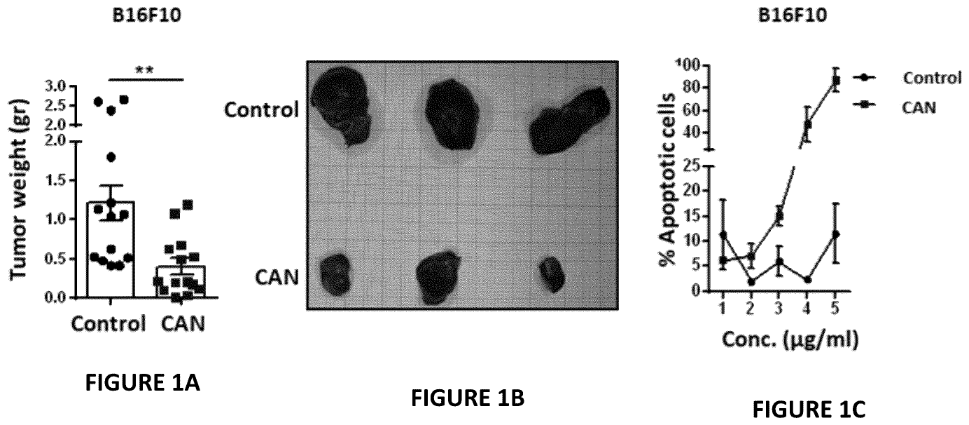

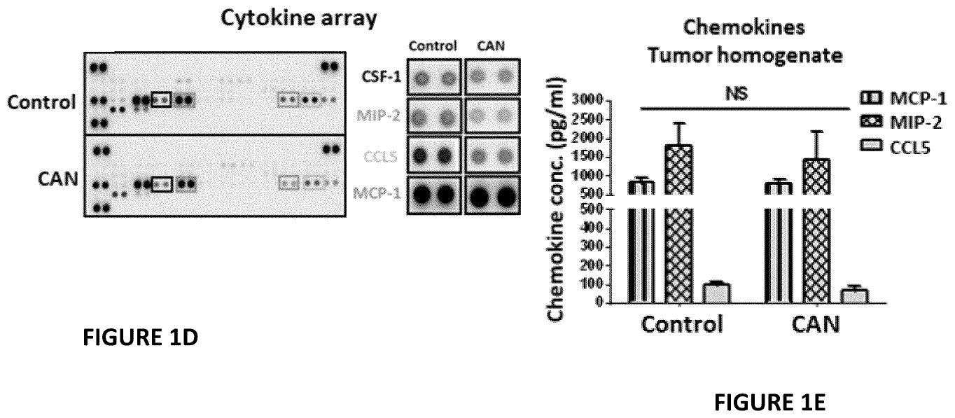

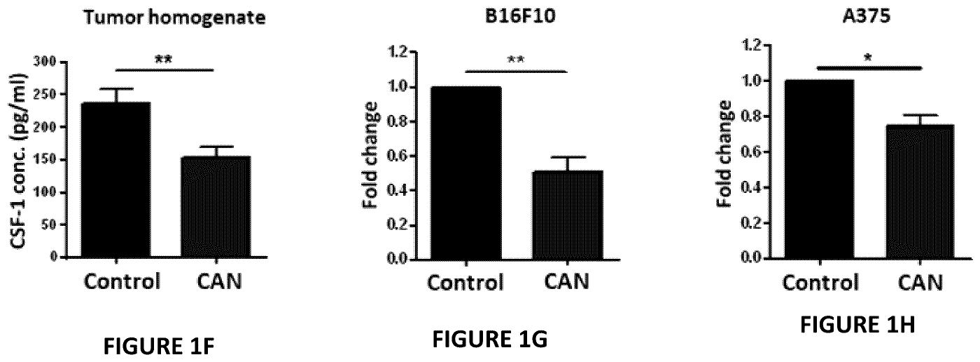

First, the inventors chose a Cannabis extract (CAN) that affects B16F10 tumor size in-vivo (FIGS. 1A-1B) but does not induce a strong apoptotic effect in-vitro (FIG. 1C). The inventors identified the major phytocannabinoids in this whole Cannabis extract using HPLC/UV according to the retention time of each specific phytocannabinoid. The major phytocannabinoids that were identified are presented in Table 1.

TABLE 1
Phytocannabinoids profile of CAN, CAN fractions and 3704 (percent
weight per weight) as measured by semi-preparative HPLC/UV.

| Phytocannabinoids | | | | | | |
|---|---|---|---|---|---|---|
| % w/w | CAN | F1 | F2 | F3 | F4 | 3704 |
| THCA | n.a | n.a | n.a | 0.74 | n.a | n.a |
| THC | 66.08 | 0.18 | 93.01 | 0.61 | n.a | 0.35 |
| CBDA | n.a | n.a | n.a | n.a | n.a | n.a |
| CBD | 0.22 | 2.6 | n.a | n.a | n.a | n.a |
| CBGA | n.a | n.a | n.a | n.a | n.a | n.a |
| CBG | 1.28 | 12.92 | n.a | 0.58 | n.a | 61.97 |
| CBDVA | n.a | n.a | n.a | n.a | n.a | n.a |
| CBDV | 0.08 | 0.53 | n.a | n.a | n.a | 0.19 |
| THCV | 0.39 | 4.07 | n.a | n.a | n.a | 0.26 |
| CBNA | n.a | n.a | n.a | n.a | n.a | n.a |
| CBN | 1.66 | 20.15 | 0.21 | n.a | n.a | 0.8 |
| CBC | 0.73 | n.a | n.a | 16.18 | n.a | 0.51 |
| CBL | n.a | n.a | n.a | n.a | n.a | n.a |
| D8-THC | n.a | n.a | n.a | n.a | n.a | n.a |
| CBCV | n.a | n.a | n.a | n.a | n.a | n.a |

The phytocannabinoid with the highest weight to weight percentage (% w/w) in the extract was THC (66%) and other phytocannabinoids were detected in lower percentages (0.08%-1.66%). When the inventors treated the B16F10 cell line with CAN, it was decided to focus on a concentration that affects the cells without inducing cell death, so that the inventors may assess differences in cytokine secretion. The inventors treated the cells with increasing concentrations of CAN and identified 2 μg/ml as the optimal concentration which does not lead to increased cell death (FIG. 1C). When B16F10 cells were treated with 2 μg/ml CAN, the inventors detected a reduction in three myeloid-related cytokines and chemokines using the cytokine screen assay (FIG. 1D): CSF-1, upregulated by cancer cells and a major differentiation and activation factor of the monocytic lineage, macrophage-inflammatory protein 2 (MIP-2), a strong chemoattractant of neutrophils, and Chemokine (C-C motif) ligand 5 (CCL5), which is involved in recruitment and involvement of tumor-promoting immune cells. Changes in the monocytic chemotactic protein 1 (MCP-1) secretion were not detected, however, MCP-1 concentrations were tested in further experiments as a positive control (FIG. 1D).

### Example 2

### CBG Reduces CSF-1 Secretion by B16F10 Cells

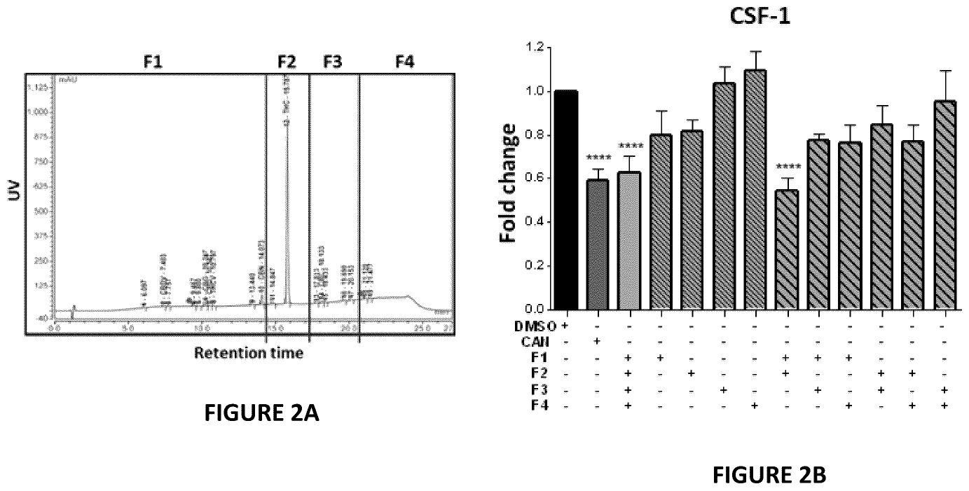

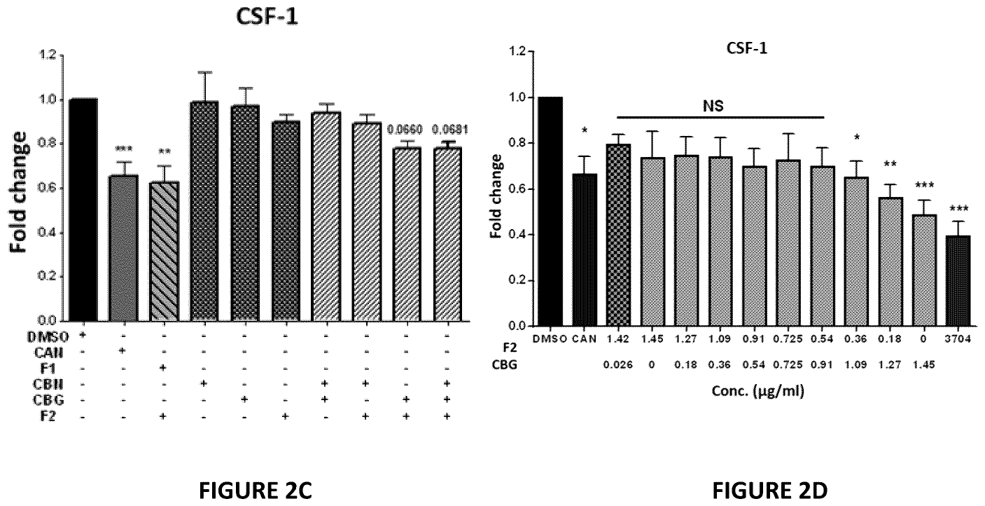

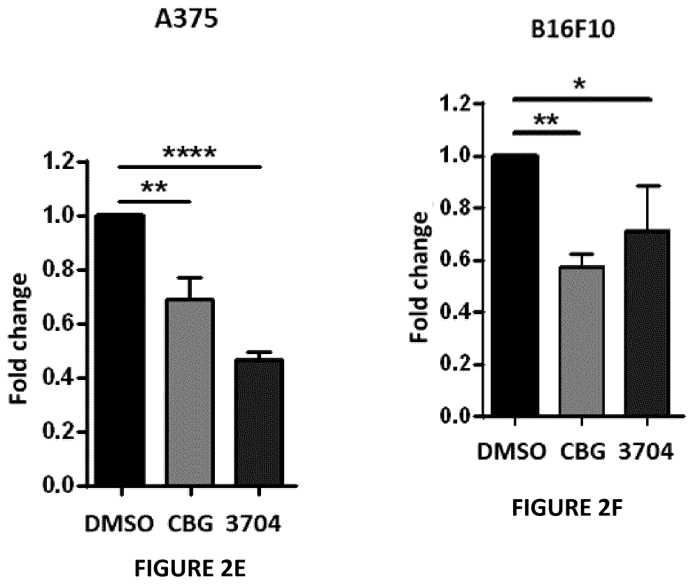

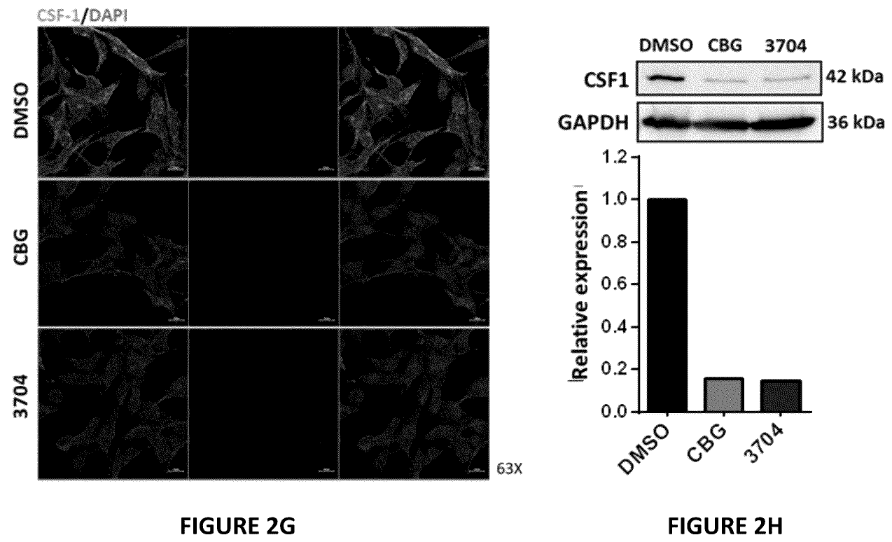

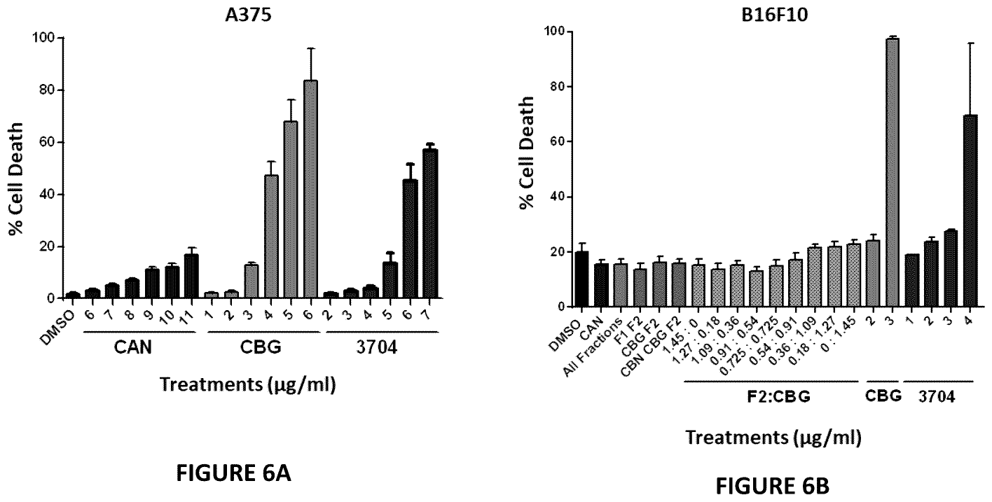

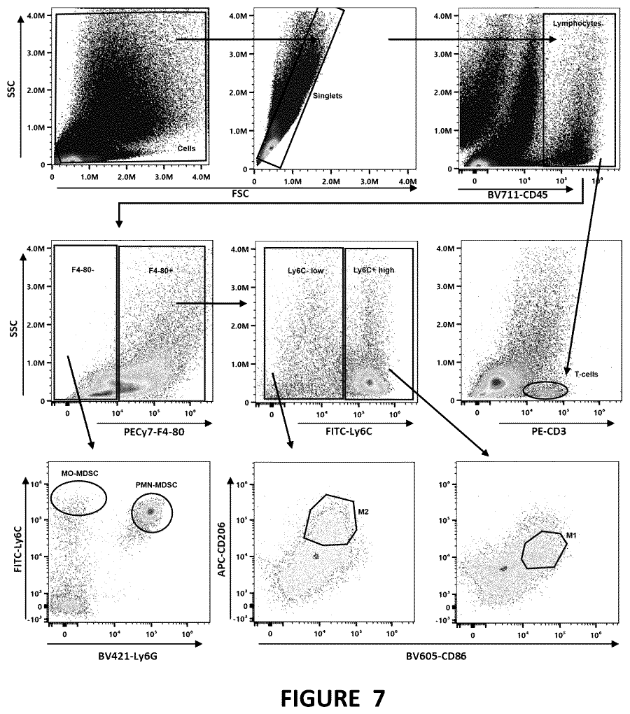

Since the inventors identified the specific reduction of CSF-1 after CAN treatment, subsequent investigation of the specific cannabinoids that are present in the whole extract and are responsible for the observed effects, was conducted. For this purpose, the inventors fractionated CAN extract into four fractions (F1-F4) (FIG. 2A). The weight-to-weight percentage of the major phytocannabinoids in each fraction is shown in Table 1. Then, B16F10 cells were treated with each fraction separately and all the possible combinations. The concentration of each fraction was normalized according to the concentration of the phytocannabinoid with the highest weight-to-weight percentage in the whole extract. The change in secreted CSF-1 concentrations was measured by ELISA (FIG. 2B). The inventors found that a combination of fractions F1 and F2 mimics the effect of the whole extract. As shown in Table 1, the phytocannabinoids with the highest weight-to-weight percentage in F1 were Cannabinol (CBN) and CBG. In F2 the major phytocannabinoid detected was THC and this fraction was almost exclusively made of THC (93%). This led us to investigate the change in CSF-1 secretion on a single molecule resolution. The inventors tested synthetic pure CBN and synthetic pure CBG in combination with F2, used as indicative of pure THC. The inventors used the same concentrations of the cannabinoids as they are present in the whole extract, and identified that CBG and F2 reduced CSF-1 secretion more efficiently than when the cells were treated separately or by any other cannabinoid combination (FIG. 2C). The addition of synthetic CBN together with CBG and F2 did not further reduce CSF-1 secretion. However, the combination of CBG and F2 was not as efficient as the whole extract or F1 and F2 combined. Therefore, the inventors tested the effect on CSF-1 secretion by different ratios of CBG and F2 (FIG. 2D). Strikingly, with increasing concentrations of CBG up to 1.45 μg/ml and decreasing concentrations of F2, CSF-1 secretion was reduced more efficiently. As a positive control, the inventors used a high-CBG chemovar, termed 3704, at the same concentration of 2 μg/ml as was used for CAN. The inventors identified a more significant reduction in CSF-1 secretion compared to the original CAN extract (FIG. 2D). rightmost column). The inventors verified these results in the A375 cell line, cells treated with 1.5 μg/ml CBG and 2 μg/ml 3704 showed reduced CSF-1 secretion in this cell line as well (FIG. 2E). Further validation was conducted by measuring CSF-1 protein levels in B16F10 cells treated with either 1.5 μg/ml CBG or 2 μg/ml 3704 using confocal microscopy or a western blot assay (FIGS. 2F-2H). Both methods showed decreased CSF-1 protein levels when cells were treated with either CBG or 3704, concluding that CBG most efficiently reduces CSF-1 protein synthesis by B16F10 cells. The toxicity of all the different treatments at the indicated concentrations was measured and no increased cell death was identified compared to DMSO (FIGS. 6A-6B).

### Example 3

### Conditioned Media from CBG-Treated B16F10 Cells Reduces MO-MDSC Expansion and Macrophage Transition

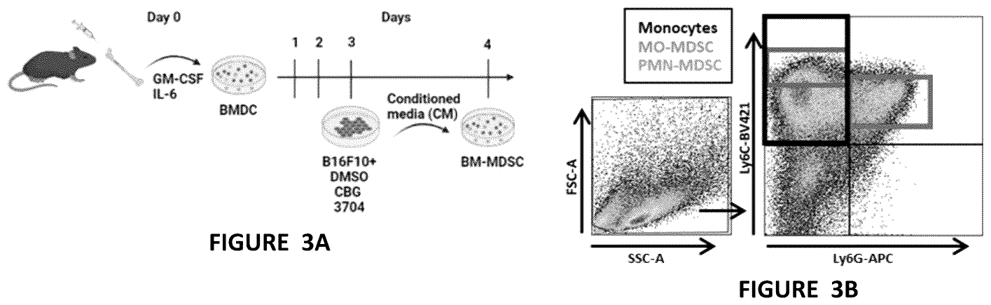

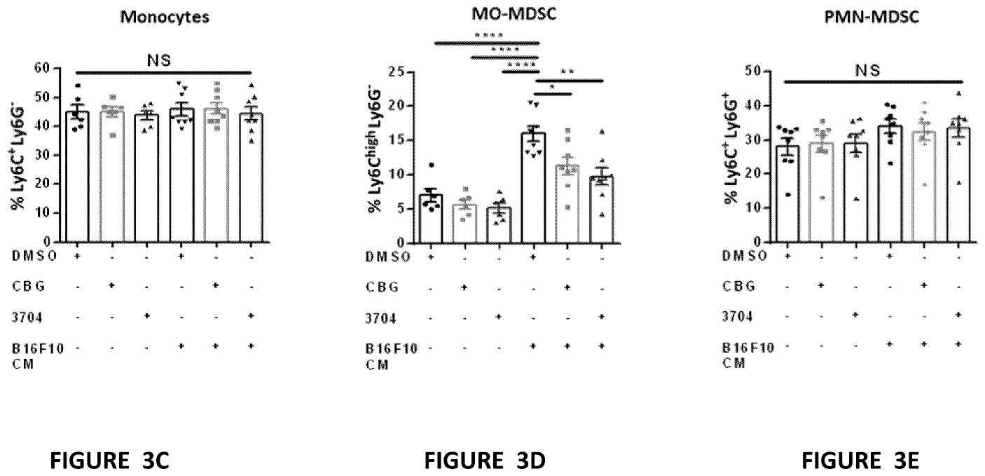

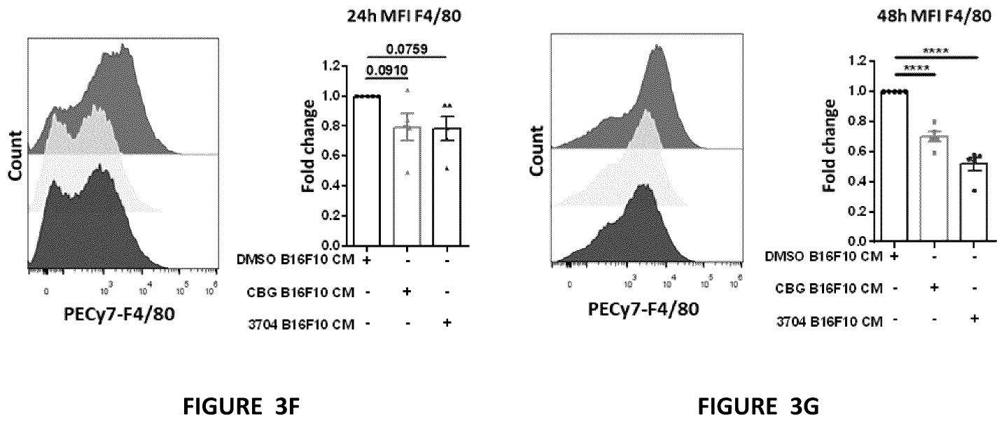

Since CSF-1 is a key regulator of differentiation and expansion of the monocyte-macrophage axis, the inventors investigated how the reduction in CSF-1 secretion by CBG-treated B16F10 might influence the myeloid subpopulation frequency distribution ex-vivo. For this purpose, the inventors isolated BMDCs from healthy WT mice and cultured the cells with cytokines to generate bone-marrow-derived myeloid-derived suppressor cells (BM-MDSCs). Then, the BM-MDSCs were treated with conditioned media (CM) from B16F10 cells cultured for 24 hours with either DMSO, CBG or 3704 (FIG. 3A). Myeloid cell frequencies were measured using flow cytometry after 24 or 48 hours and the gating strategy is shown in FIG. 3B. As a control, the inventors also treated the BM-MDSCs with control growth media that were incubated without cells with the same concentrations of DMSO, CBG and 3704 treatments as they were added to the B16F10 cells. The inventors found that the overall percentage of monocytes (Ly6C⁺/Ly6G⁻ cells) did not change when BM-MDSCs were treated with the different B16F10 CM nor with control growth media (FIG. 3C). However, the inventors identified a significant increase in the frequency of the Ly6C^{high}/Ly6G⁻ MO-MDSC subpopulation, when BM-MDSCs were treated with CM from B16F10 (FIG. 3D). Moreover, when BM-MDSCs were treated with CM from B16F10 treated with either CBG or 3704, the inventors detected a significant decrease in MO-MDSC percentages compared to B16F10 treated with DMSO (FIG. 3D). The effect was specific to the subset of MO-MDSCs as PMN-MDSC frequencies (LyC6⁺/Ly6G⁺ [26]) were not affected by any treatment (FIG. 3E).

During tumor progression, monocytes and MO-MDSC can differentiate into anti-inflammatory macrophages that promote the tolerogenic TME. The differentiation of MO-MDSCs to anti-inflammatory macrophages in the TME is mainly mediated by tumor-secreted CSF-1. Therefore, the inventors investigated whether reduced CSF-1 secretion by CBG-treated B16F10 cells affects the transition of MO-MDSCs to F4/80⁺ macrophages. The inventors measured the mean fluorescent intensity (MFI) of F4/80 on Ly6C⁺ cells treated with B16F10 CM for 24 (FIG. 3F) and 48 hours (FIG. 3G). The inventors identified a reduction in the F4/80 MFI in Ly6C⁺ cells when CM from B16F10 cells treated with CBG or 3704 was added to the cells compared to DMSO. This trend was apparent after 24 hours and was statistically significant after 48 hours.

To validate the change in secretion observed by the B16F10 cell line, the four above indicated cytokines and chemokines were measured in B16F10 tumors from mice that were treated either with a control vehicle or with CAN, using ELISA. The inventors were able to detect only a reduction in the concentration of CSF-1 in tumors of CAN-treated mice (FIGS. 1E-1F). In-vitro validation of CSF-1 secretion by B16F10 cell line and another type of melanoma cell line, A375 human melanoma cells, showed the reduced secretion of CSF-1 by CAN-treated cells as well (FIGS. 1G-1H). The optimal concentration of CAN for A375 was assessed in a similar manner to B16F10 and found as 6 μg/ml in-vitro (FIG. 6A, leftmost columns).

### Example 4

### MO-MDSCs Express Lower Levels of iNOS Leading to Restored CD8+ T-Cell Activation After Treatment with CM from B16F10 Treated with CBG

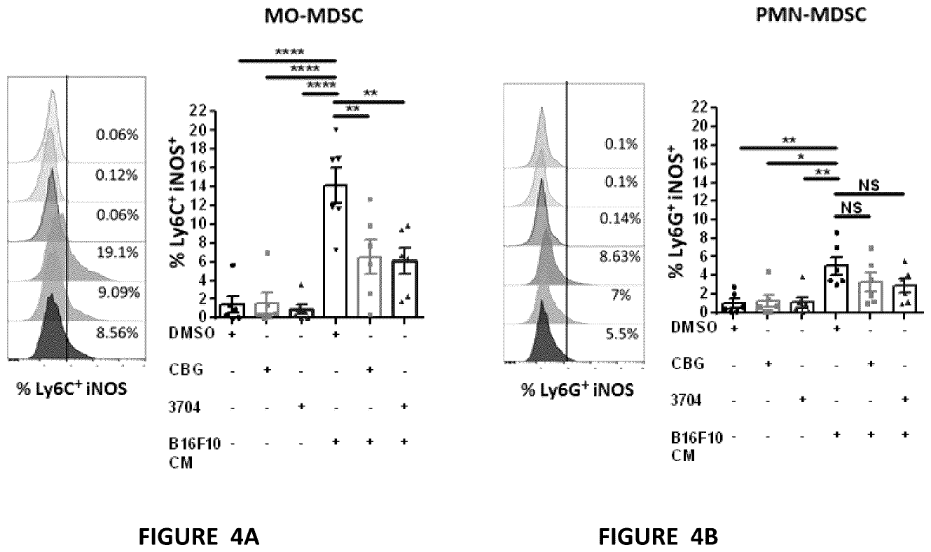

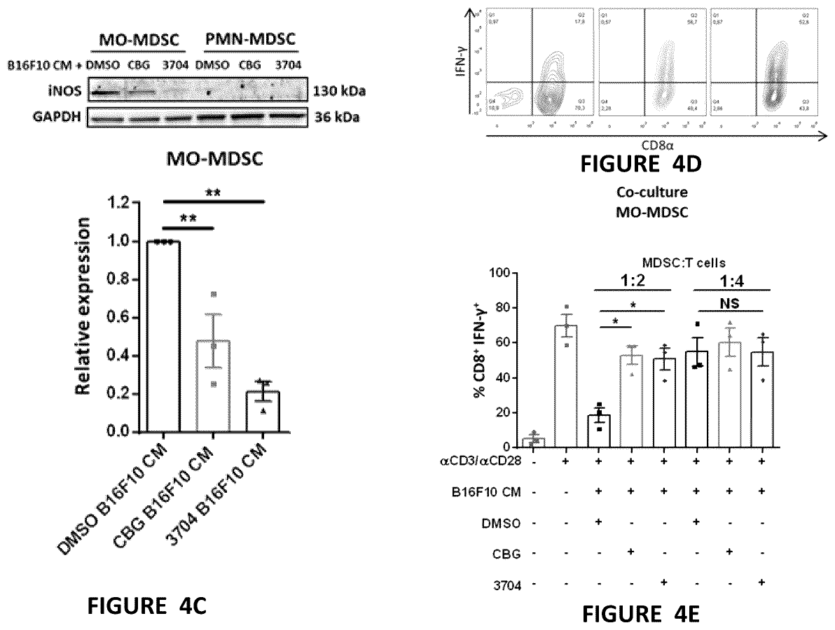

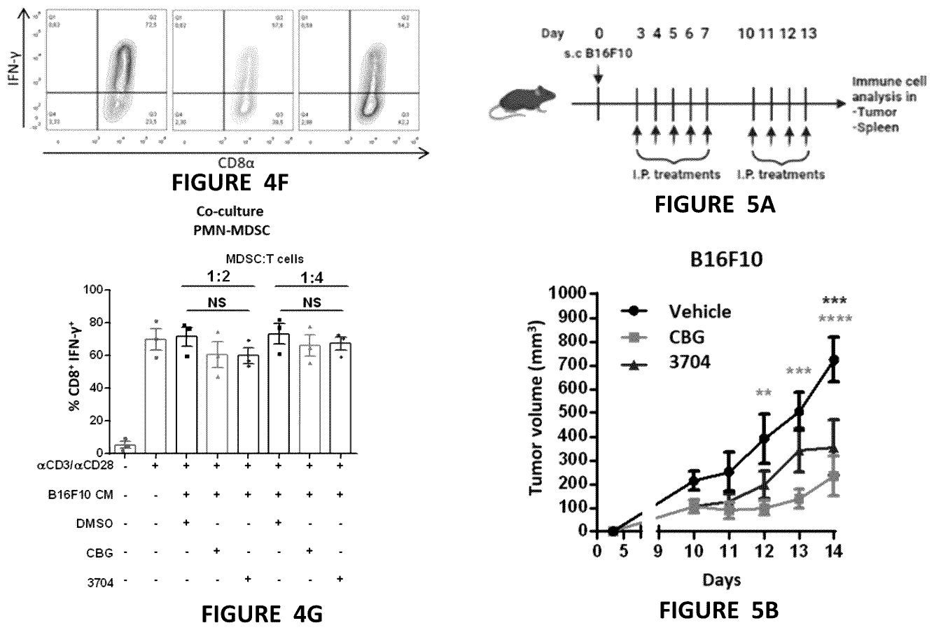

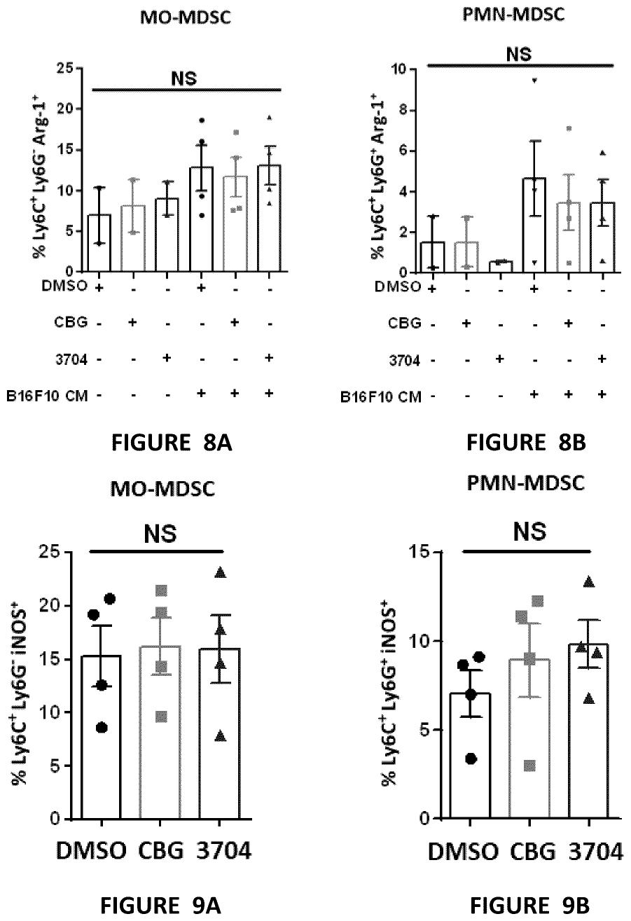

The current results showed that reduced CSF-1 secretion by B16F10 cells treated with CBG or 3704 specifically affects the expansion of Ly6C^{high} MO-MDSC and reduces the transition to F4/80 expressing macrophages ex-vivo. The inventors hypothesized the reduced MO-MDSC expansion is associated with decreased immunosuppressive properties as well. To test this, the inventors generated BM-MDSCs and treated them with control growth media or CM, and after 24 hours the expression of the immunosuppressive markers Arginase-1 (Arg-1) and inducible nitrite oxide synthase (iNOS) by the BM-MDSC subpopulations was assessed using flow cytometry. Arg-1 expression in MO-MDSCs and PMN-MDSCs did not change significantly when cells were treated with CM from B16F10 cells compared to when cells were treated with control growth media (FIG. 8). In addition, when BM-MDSCs were exposed to CM of CBG-or 3704-treated B16F10, the inventors did not detect a significant difference in Arg-1 expression compared to CM of DMSO-treated B16F10 cells in neither of the MDSC subpopulations (FIG. 8). However, the expression of iNOS by BM-MDSCs increased significantly for the subset of MO-MDSCs when the CM of DMSO-treated B16F10 cells was added to the BM-MDSCs (FIG. 4A, gray). The addition of CM of CBG-or 3704-treated B16F10 cells significantly decreased iNOS expression in specifically MO-MDSC compared to CM of DMSO treated B16F10 cells (FIG. 4A, yellow, red). iNOS expression also increased by PMN-MDSCs when CM of DMSO-treated B16F10 cells was added (FIG. 4B, gray), although not to the same extent as the MO-MDSC subset. Additionally, the inventors did not detect a significant decrease in iNOS expression by PMN-MDSC, when exposed to CM of CBG- or 3704-treated B16F10 cells compared to CM of DMSO-treated B16F10 cells (FIG. 4B). To exclude direct effects of CBG and 3704 on iNOS expression by MDSCs, the inventors generated BM-MDSCs as described and added medium from untreated B16F10 to the cells together with either DMSO, CBG or 3704. iNOS expression was measured in each subpopulation by flow cytometry and the inventors did not detect any significant differences (FIGS. 9A-9B), indicating that the observed effect is mediated through the change in tumor cytokine secretion in the TME.

For further validation, the inventors sorted the generated BM-MDSCs and sorted them into MO-MDSC and PMN-MDSC subsets, then measured iNOS expression in each subpopulation by western blot (FIG. 4C). The inventors identified a reduction of iNOS expression specifically in MO-MDSCs when CM from CBG- or 3704-treated B16F10 cells was added to the BM-MDSCs compared to CM of DMSO-treated B16F10 cells. Here again, PMN-MDSC expressed very low levels of iNOS.

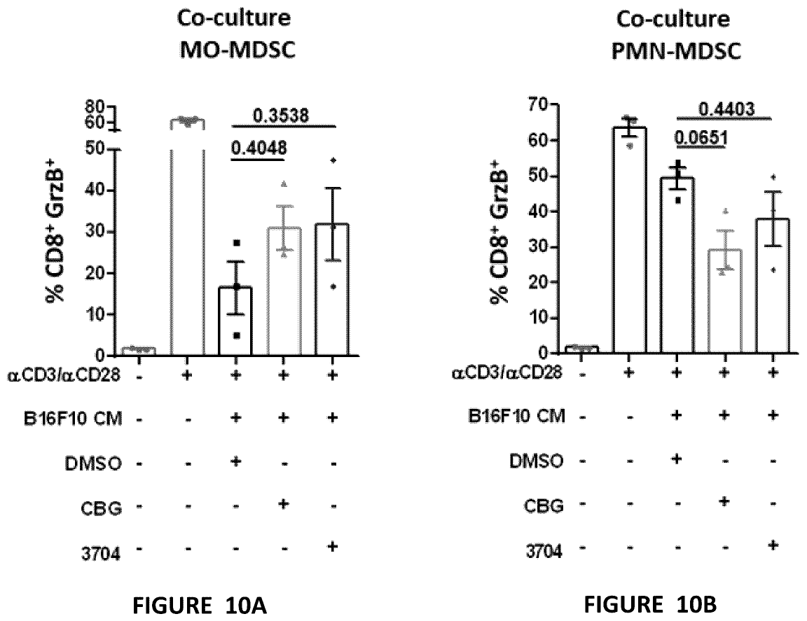

Many studies have shown the involvement of increased iNOS expression by MDSCs in suppressing CD8+ T-cell activation in the TME. Since CM from CBG- or 3704-treated B16F10 cells reduced iNOS expression specifically in MO-MDSCs, the inventors tested the ability of sorted and treated BM-MDSC subpopulations to restore CD8+ activation ex-vivo. For this purpose, the inventors established a co-culture experiment of CD8+ T-cell and MO-MDSCs or PMN-MDSCs. After 48 hours of co-incubation, CD8+ T-cells were assessed for intracellular expression of GranzymeB (GrzB) and Interferon-y (IFN-γ) using flow cytometry (FIGS. 4D-4G). GrzB expression levels were lowered in T-cells co-cultured with MO-MDSCs exposed to CM of DMSO-treated B16F10 cells (FIG. 10A). Moreover, the inventors detected a trend of restored GrzB expression when T-cells were co-cultured with MO-MDSCs exposed to CM of CBG- or 3704-treated B16F10 cells (FIG. 10A). Co-culture of T-cells with PMN-MDSCs exposed to CM of DMSO-treated B16F10 cells did not result in reduced GrzB expression (FIG. 10B, gray) and a trend of reduced GrzB expression was detected when T-cells were co-cultured with PMN-MDSC exposed to CM of CBG-or 3704-treated B16F10 cells (FIG. 10B). The inventors found that MO-MDSC specifically exposed to CM of DMSO-treated B16F10 cells suppressed significantly CD8+T-cell IFN-γ expression compared to PMN-MDSCs that received the same treatment (FIGS. 4D-4G). Additionally, when CM of CBG- or 3704-treated B16F10 was added to MO-MDSCs, the inventors detected a restoration of CD8+ IFN-γ expression in a dose-dependent manner (FIG. 4E). In contrast, CD8+T-cells co-cultured with PMN-MDSCs showed no significant changes with either of the treatments (FIG. 4G).

### Example 5

### CBG Treatment Reduces B16F10 Tumor Progression and M2 Macrophage Frequencies in the Tumors

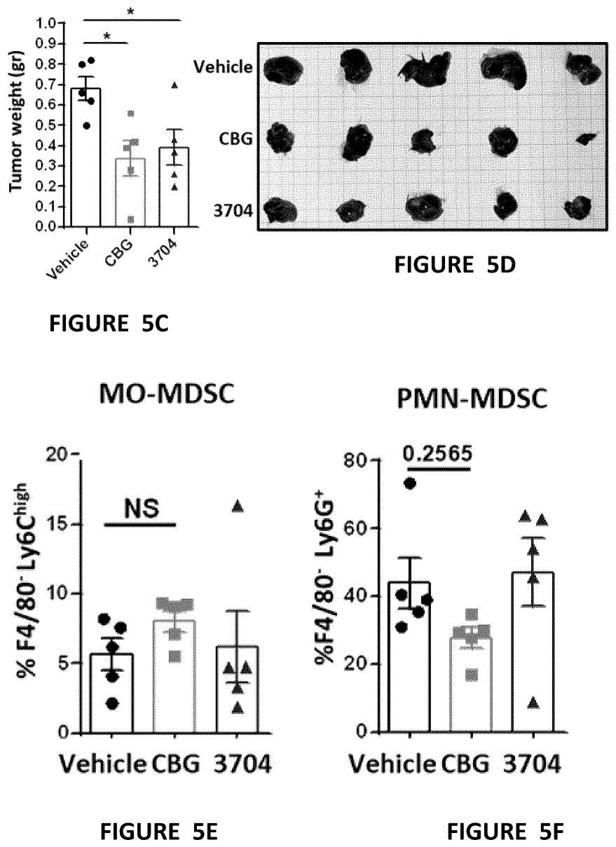

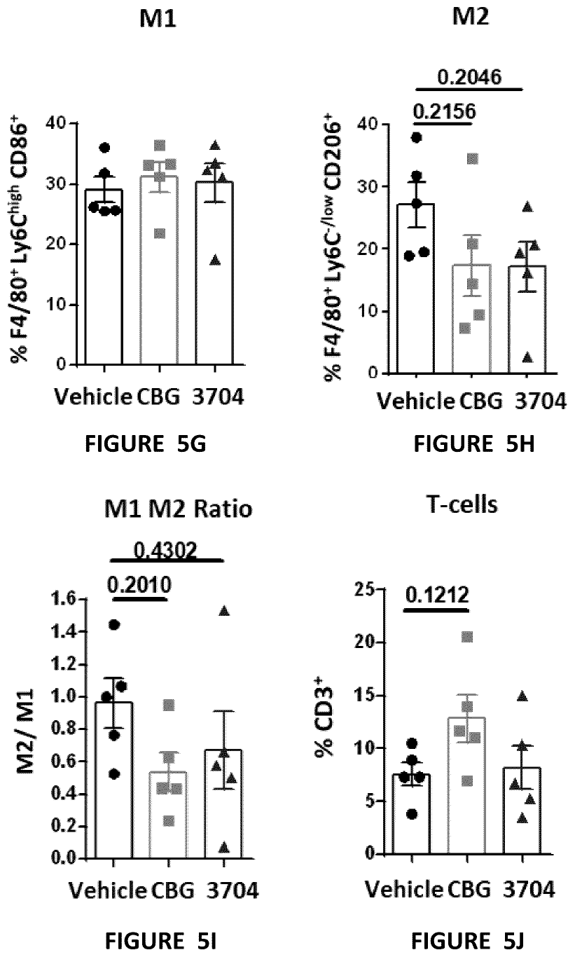

As the next step, the inventors measured B16F10 tumor development in WT mice that were treated with CBG or 3704, and investigated the frequencies of the myeloid cell subpopulations in the tumors. For this purpose, the inventors injected subcutaneously B16F10 cells into the mice and after 3 days treated intraperitoneally with injections of CBG or 3704 (FIG. 5A); mice received overall nine injections. Tumor volume was measured daily between day 10 and 14, and it was significantly lower in the CBG-treated group already at day 12, and significantly lower in the 3704-treated group on day 14 compared to the vehicle-treated group (FIG. 5). Tumors were harvested on day 14 from the initial B16F10 injection and weighed. The tumors that were dissected from the CBG- and 3704-treated mice weighed significantly less compared to tumors treated with vehicle (FIGS. 5C-5D). To assess the subpopulations of myeloid cells in the tumors, the inventors prepared single-cell suspension from the tumors and stained them with specific extracellular markers (FIGS. 5E-5H). Treatments did not affect the frequencies of MO-MDSC and PMN-MDSC subpopulations (FIGS. 5E-5F), however, there was a trend of a decreased frequency in M2 macrophages in the CBG- and 3704-treated groups relative to vehicle control (FIG. 5H). The inventors analyzed the ratio between the pro-inflammatory M1 macrophages and the anti-inflammatory M2 macrophages (FIG. 5I) and found a trend of decreased ratio in the CBG- and 3704-treated groups relative to vehicle control, indicative of a less immunosuppressive TME. In addition, the inventors stained for the general T-cell marker CD3, and found a trend of increase in T-cells frequencies in the CBG- and 3704-treated groups relative to vehicle control, possibly indicative of higher cytotoxic T-cell infiltration into these tumors (FIG. 5J).

### Discussion

The therapeutic strategy of TME modulation is gaining increasing attention in the field of anticancer treatment. Immune checkpoint blockade therapy that blocks inhibitory receptors expressed on the surface of immune cells has been in use for oncology patients over the last decade, alone or in combination with chemotherapy. Inhibitory antibodies such as anti-programmed cell death protein 1 (PD1) or anti-cytotoxic T lymphocyte antigen 4 (CTLA4) disrupt the negative regulation between cancer cells and cytotoxic T-cells to harness the cytotoxic abilities of the immune system. However, there are several molecular mechanisms allowing resistance to blockade therapy. Recent studies revealed it is insufficient to stimulate the cytotoxic T-cells with immune checkpoint blockade therapy. rather, concurrently targeting immunosuppressive cells such as regulatory myeloid cells in the TME is needed to fully utilize the potential of the host immune system to suppress the progression of cancer. The inventors therefore set out to examine whether Cannabis extracts can immune-regulate the regulatory myeloid cells in the TME.

The inventors found a Cannabis extract able to specifically and significantly reduce the secretion of CSF-1 in-vitro and in-vivo. Many clinical trials have been focusing on modulating immunosuppressive cells by the targeted depletion of tumor secreted cytokines and as CSF-1 is a major modulator of regulatory myeloid cells in the TME, targeting the CSF-1 axis has been under extensive investigation. CSF-1 is highly expressed by several tumor types, and this together with the expression of the CSF-1 receptor by macrophages in the tumors are associated with poor prognosis and survival; emphasizing the relevance of targeting this cytokine. Moreover. CSF-1 is specific to the monocyte-macrophage axis, therefore its depletion does not affect other immune cell populations, possibly making it more tolerable, as was already confirmed by early clinical data. The current findings provide a new tool for targeting this major tumor secreted cytokine, with highly relevant clinical applications.

The inventors succeeded to purify and identify from the whole-extract a single cannabinoid, CBG, able to reduce CSF-1 secretion more efficiently than the psychoactive THC, the major cannabinoid in the extract. CBG is a non-psychoactive cannabinoid, which makes it more suitable for medical application. CBG exhibited different affinities than THC or CBD to cannabinoid receptors and was shown to uniquely target adrenergic and serotonin receptors. However, only a few studies investigated the pharmacological effects of CBG and none tested its effects on the TME. To verify our findings for CBG, the inventors utilized a high-CBG chemovar termed 3704 and found it was as efficient as pure CBG and more efficient than the high-THC chemovar. Medical Cannabis has been classically divided into three phenotypic chemovar groups according to its content of THC and CBD: Type I which is THC-predominant. Type II in which the two are balanced and Type III which is CBD-predominant. As CBG serves as the precursor for the abundant phytocannabinoids, it is normally found in very low quantities in medical Cannabis. Cannabis chemovars that lack the enzymes that convert the acid forms of CBG to either THC or CBD can accumulate CBG and are consequently CBG-rich, and now known as Type IV. Importantly, to date, high-CBG chemovars are not commercially available and patients are prescribed medical Cannabis based on the THC:CBD ratio. In this work, the inventors used for the first time a non-psychoactive Type IV chemovar and proved its supremacy over the highly used psychoactive Type I.

Regulatory myeloid cells comprise several immunosuppressive cells types, namely MO-MDSC, PMN-MDSC and M2-like macrophages. Extensive research of the past years showed that each subpopulation is influenced by different cytokines and chemokines. The current results show that the specific reduction of CSF-1 secretion by CBG- and 3704-treated melanoma cells reduces the expansion of specifically the MO-MDSC subpopulation, without affecting the frequency of PMN-MDSCs. Moreover, the inventors found that this treatment reduces the transition to macrophages, as well. Each MDSC subpopulation possesses a different degree of immunosuppressive capabilities. Studies show although PMN-MDSC are more abundant in the TME, MO-MDSC encounter a stronger immunosuppressive phenotype than PMN-MDSC [47]. Moreover, a higher proportion of MO-MDSCs was previously found in peripheral blood from melanoma patients, emphasizing the importance of targeting the MO-MDSC subpopulation in the TME. Many immunosuppressive markers that are upregulated by MDSCs have been discovered and the inventors focused on the prominent Arg-1 and INOS. The inventors found that the CM of B16F10 cells treated with DMSO increased specifically the iNOS expression by MO-MDSC and not by PMN-MDSC, supporting past findings of stronger immunosuppressive properties of MO-MDSC compared to PMN-MDSCs. Moreover, CM of B16F10 treated with CBG or 3704 reduced the iNOS expression specifically in MO-MDSCs, again underlying the importance of CSF-1 in modulating the immunosuppressive properties of the monocyte lineage in the TME. Co-culture experiments demonstrated again the difference in immunosuppressive capacity of MDSC subpopulations, only MO-MDSC were able to suppress CD8+ T-cell activity. Accordingly, only treatment of MO-MDSC with CM of B16F10 cells treated with CBG or 3704 restored CD8+ T-cell activity. In an in-vivo model, CBG or 3704 treated mice had no significant changes in MDSCs frequencies. However, the inventors did detect trend of reduced frequencies of M2-macrophages and a reduced M2/M1 ratio, which aligns with the current in-vitro results that showed the reduced transition to macrophages.

The inventors found reduced tumor progression in mice treated with CBG or 3704. Future studies will need to verify this is due to an effect on the TME and not directly on the tumor cells. Future research should also examine the combined effect of CBG with immune checkpoint blockade therapies such as anti-PD-1. A possible outcome of co-treating will be the generation of more cytotoxic CD8+ T-cells that may exert better anticancer effects.

Although the connection between the endocannabinoid system and the immune system is well established, only a few studies focused on investigating its involvement in TME modulation. Because of the immunoregulatory properties of cannabinoids, the endocannabinoid system was previously suggested to play a fundamental role in shaping the TME and influencing tumor progression. One study has shown the endocannabinoid 2-arachidonoylglycerol (2-AG) exhibits direct antitumor effects but also promotes an immunosuppressive microenvironment by increasing the suppressive immune cell population of MDSCs. Another study focused on the effect of CBD on cytokine secretion in triple-negative breast cancer. Conditioned media from CBD-treated cancer cells lowered the levels of Granulocyte-macrophage (GM)-CSF, a colony-stimulating factor from the same family as CSF-1, and CCL3, which are important for macrophage recruitment and activation. These studies together with the current results, support the potential of cannabinoids in modulating the TME in a variety of cancers. Moreover, the inventors bring support for the utility of the minor cannabinoids such as CBG in therapeutic settings.

Medical Cannabis is already being prescribed to cancer patients, primarily as palliative care meant to alleviate pain, relieve nausea and stimulate appetite. The inventors have previously shown medical Cannabis treatment is generally safe for oncology patients. However, there is a huge variety between different medical Cannabis chemovars in their phytocannabinoid composition. In another study, the inventors found Cannabis consumption was associated with worsening the success rate of blockade therapy. It is possible that these patients received medical Cannabis chemovars that were THC- or CBD-rich, with very little CBG, indicating the pressing need for precision medicine that uses the right treatment. Therefore, it is of great importance to first identify the specific active molecules in the whole extract. The current findings have immediate practical implications, current treatment protocols that are already in combination with medical Cannabis as palliative care can select the CBG-rich chemovars in combination with immune checkpoint blockade therapy, making it more effective, and providing patients with antitumor properties in addition to the palliative ones.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. A method for increasing the therapeutic efficacy of an immune checkpoint inhibitor in a subject treated therewith, the method comprising administering to said subject being treated with said immune checkpoint inhibitor a therapeutically effective amount of a pharmaceutical composition comprising cannabigerol (CBG) or any salt thereof, thereby increasing the therapeutic efficacy of an immune checkpoint inhibitor in the subject.

2. The method of claim 1, wherein said subject is afflicted with cancer.

3. The method of claim 1, wherein said pharmaceutical composition consists essentially of CBG.

4. A method for treating cancer in a subject in need thereof, the method comprising administering to said subject CBG and an immune checkpoint inhibitor, thereby treating cancer in the subject.

5. The method of claim 4, wherein said administering is at a synergistically effective amount.

6. The method of claim 1, wherein said CBG is present as a highly purified extract of Cannabis.

7. The method of claim 1, wherein said CBG is synthetically produced.

8. The method of claim 4, wherein said treating comprises: reducing weight and/or volume of a tumor, increasing % of cancer cells undergoing apoptosis or cell death, reducing expression and/or secretion levels of colony stimulating factor 1 (CSF-1) from cancer cells, reducing number or abundance of myeloid-derived suppressor cells (MDSCs), M2-like tumor-associated macrophages, or both, increasing the number of cytotoxic CD8⁺ T cells, or any combination thereof, in said subject.

9. The method of claim 8, wherein said cytotoxic CD8+ T cells are expressing, secreting, or both, interferon gamma (INF-γ).

10. The method of claim 1, wherein said administering comprises administering said CBG and said immune checkpoint inhibitor separately or concomitantly.

11.-13. (canceled)

14. A kit comprising:
a. CBG; and
b. an immune checkpoint inhibitor.

15. The kit of claim 14, further comprising instructions for: (i) mixing said CBG and immune checkpoint inhibitor; (ii) administering the mixed CBG and immune checkpoint inhibitor of (i) to a subject in need thereof; or (iii) both (i) and (ii).

16. (canceled)

17. The method of claim 5, wherein said CBG is present as a highly purified extract of Cannabis.

18. The method of claim 5, wherein said CBG is synthetically produced.

19. The method of claim 4, wherein said administering comprises administering said CBG and said immune checkpoint inhibitor separately or concomitantly.
